(19) United States
(12) Patent Application Publication
(10) Pub. No.: US 2011/0200626 A1
(43) Pub. Date: Aug. 18, 2011

(54) IQGAP3 EPITOPE PEPTIDES AND VACCINES CONTAINING THE SAME

(75) Inventors: Takuya Tsunoda, Kanagawa (JP); Ryuji Ohsawa, Kanagawa (JP); Sachiko Yoshimura, Kanagawa (JP); Tomohisa Watanabe, Kanagawa (JP)

(73) Assignee: Oncotherapy Science, Inc., Kanagawa (JP)

(21) Appl. No.: 12/997,517

(22) PCT Filed: Jun. 10, 2009
(86) PCT No.: PCT/JP2009/002613

§ 371 Date: Apr. 28, 2011

Related U.S. Application Data
(60) Provisional application No. 61/060,538, filed on Jun. 11, 2008.

(51) Int. Cl. A61K 39/00; C12N 15/87; C12N 5/0783; C07K 7/06; C12N 5/071; A61K 38/08; C07H 21/00; A61K 31/7088; A61P 35/00; A61P 37/04

(52) U.S. Cl. 424/185.1; 435/455; 435/325; 530/328; 514/19.2; 536/23.1; 514/19.3; 514/44 R

(57) ABSTRACT

Peptide vaccines against cancer are described herein. In particular, the present invention describes epitope peptides derived from IQGAP3 that elicit CTLs. The present invention also provides established CTLs that specifically recognize HLA-A24 or HLA-A02 positive target cells pulsed with the peptides. Antigen-presenting cells and exosomes that present any of the peptides, as well as methods for inducing antigen-presenting cells are also provided. The present invention further provides pharmaceutical agents containing the IQGAP3 polypeptides or polynucleotides encoding thereof, as well as exosomes and antigen-presenting cells as active ingredients. Furthermore, the present invention provides methods for treating and/or prophylaxis of (i.e., preventing) cancers (tumors), and/or prevention of postoperative recurrence thereof, as well as methods for inducing CTLs, methods for inducing anti-tumor immunity, using the IQGAP3 polypeptides, polynucleotides encoding the polypeptides, exosomes or antigen-presenting cells presenting the polypeptides, or the pharmaceutical agents of the present invention. The cancers to be targeted include, but are not limited to, renal, esophageal, gastric, lung, breast, bladder and pancreatic cancer.

### PRIORITY

The present application claims the benefit of U.S. Provisional Application No. 61/060,538, filed Jun. 11, 2008, the entire content of which is incorporated by reference herein.

### TECHNICAL FIELD

The present invention relates to the field of biological science, more specifically to the field of cancer therapy. In particular, the present invention relates to novel peptides that are extremely effective as cancer vaccines, and drugs for treating and preventing tumors.

### BACKGROUND ART

It has been demonstrated that CD8 positive CTLs recognize epitope peptides derived from the tumor-associated antigens (TAAs) found on major histocompatibility complex (MHC) class I molecules, and then kill the tumor cells. Since the discovery of the melanoma antigen (MAGE) family as the first example of TAAs, many other TAAs have been discovered, primarily through immunological approaches (Boon T. Int J Cancer 54: 177-180, 1993; Boon T, and van der Bruggen P. J Exp Med 183: 725-729, 1996; van der Bruggen P, et al. Science 254: 1643-1647, 1991; Brichard V, et al. J Exp Med 178: 489-495, 1993; Kawakami Y, et al. J Exp Med 180: 347-352, 1994). Some of these TAAs are currently undergoing clinical development as immunotherapeutic targets.

Identification of new TAAs capable of inducing potent and specific anti-tumor immune responses, warrants further development and clinical application of peptide vaccination strategies for various types of cancer (Harris C C, J Natl Cancer Inst 1996 Oct. 16, 88(20): 1442-55; Butterfield L H et al., Cancer Res 1999 Jul. 1, 59(13): 3134-42; Vissers J L et al., Cancer Res 1999 Nov. 1, 59(21): 5554-9; van der Burg S H et al., J Immunol 1996 May 1, 156(9): 3308-14; Tanaka F et al., Cancer Res 1997 Oct. 15, 57(20): 4465-8; Fujie T et al., Int J Cancer 1999 Jan. 18, 80(2): 169-72; Kikuchi M et al., Int J Cancer 1999 May 5, 81(3): 459-66; Oiso M et al., Int J Cancer 1999 May 5, 81(3): 387-94). To date, there have been several reports of clinical trials using these tumor-associated antigen derived peptides. Unfortunately, only a low objective response rate has been observed in these cancer vaccine trials so far (Belli F et al., J Clin Oncol 2002 Oct. 15, 20(20): 4169-80; Coulie P G et al., Immunol Rev 2002 October, 188: 33-42; Rosenberg S A et al., Nat Med 2004 September, 10(9): 909-15).

TAAs which are indispensable for proliferation and survival of cancer cells are valiant as targets for immunotherapy, because the use of such TAAs may minimize the well-described risk of immune escape of cancer cells attributable to deletion, mutation, or down-regulation of TAAs as a consequence of therapeutically driven immune selection.

IQGAPs, IQ motif containing GTPase activating proteins, are known to regulate many actin cytoskeleton-based activities via interactions with Cdc42, Rac and RhoA. All of the IQGAP family proteins contain conserved domains, including a RasGAP-related domain, an IQ motif, and a calponin homology domain. IQGAPs are known as the effector of activated Rac1 and Cdc42 and directly interact with actin filaments. A recent search for sequences in chromosome 1 homologous to IQGAP1 let to the identification of IQGAP3 (GenBank Accession No: NM_{—}178229, SEQ ID NO: 153 encoding SEQ ID NO: 154) as a novel member of the IQGAP family (Wang S et al., J Cell Sci 2007 Feb. 15, 120: 567-77). In addition, through gene expression profile analysis using a genome-wide cDNA microarray containing 23,040 genes, IQGAP3 was identified as a novel molecule up-regulated in gastric cancer (Jinawath N et al., AACR 2006). In fact, IQGAP3 has been shown to be up-regulated in several cancer cells, including, for example bladder cancer (WO2006/085684), renal cell carcinoma (WO2007/013575), lung cancer (WO2004/031413 and WO2007/013665), esophageal cancer (WO2007/013671), pancreatic cancer (WO2004/031412) and breast cancer, the disclosures of which are incorporated by reference herein. From the expression analysis in human normal tissues, IQGAP3 transcripts were modestly detected in testis, small intestine and colon. Accordingly, IQGAP3 is considered to be a suitable target for cancer immunotherapy and epitope peptides derived therefrom may be expected to serve as cancer immunotherapeutics effective in the treatment of a wide array of cancer types.

### SUMMARY OF INVENTION

The present invention is based, in part on the discovery, of IQGAP3.as a suitable target of immunotherapy Because TAAs are generally perceived by the immune system as “self” and therefore often have no innate immunogenicity, the discovery of appropriate targets is of extreme importance. Recognizing that IQGAP3 has been identified as up-regulated in cancers tissues such as bladder, kidney, lung, esophagus, stomach, breast, and pancreas, the present invention targets this cell-division-cycle-associated 1 (CDA1) protein (IQGAP3) (SEQ ID NO: 154 encoded by the gene of GenBank Accession No. NM_{—}178229 (SEQ ID NO: 153)) for further analysis. In particular, IQGAP3 gene products containing epitope peptides that elicit CTLs specific to the corresponding molecules were selected. Peripheral Blood Mononuclear Cells (PBMC) obtained from a healthy donor were stimulated using HLA-A*24 and HLA-A*02 binding peptides derived from IQGAP3. CTLs that specifically recognize HLA-A24 or HLA-A02 positive target cells pulsed with the respective candidate peptides were established, and HLA-A24 or HLA-A02 restricted epitope peptides that can induce potent and specific immune responses against IQGAP3 expressed on the surface of tumor blood vessels were identified. These results demonstrate that IQGAP3 is strongly immunogenic and the epitopes thereof are effective targets for tumor immunotherapy.

Accordingly, it is an object of the present invention to provide peptides having CTL inducibility as well as an amino acid sequence selected from the group consisting of SEQ ID NOs: 2, 4, 7, 21, 25, 29, 32, 35, 37, 40, 49, 53, 55, 56, 57, 62, 63, 67, 75, 85, 99, 101, 111, 114, 121, 125, 130, 139, 140, 141, 142, 143, 145, 148 and 150. In addition, the present invention contemplates modified peptides, having an amino acid sequence of SEQ ID NOs: 2, 4, 7, 21, 25, 29, 32, 35, 37, 40, 49, 53, 55, 56, 57, 62, 63, 67, 75, 85, 99, 101, 111, 114, 121, 125, 130, 139, 140, 141, 142, 143, 145, 148 and 150, wherein one, two or more amino acids are substituted or added, so long as the modified peptides retain the original CTL inducibility.

When administered to a subject, the present peptides are presented on the surface of antigen-expressing cells and then induce CTLs targeting the respective peptides. Therefore, it is an object of the present invention to provide antigen-presenting cells and exosomes presenting any of the present peptides, as well as methods for inducing antigen-presenting cells.

An anti-tumor immune response is induced by the administration of the present IQGAP3 polypeptides or polynucleotide encoding the polypeptides, as well as exosomes and antigen-presenting cells which present the IQGAP3 polypeptides. Therefore, it is an object of the present invention to provide pharmaceutical agents containing the polypeptides of the present invention or polynucleotides encoding them, as well as the exosomes and antigen-presenting cells containing such as their active ingredients. The pharmaceutical agents of the present invention find particular utility as vaccines.

It is a further object of the present invention to provide methods for the treatment and/or prophylaxis of (i.e., preventing) cancers (tumors), and/or prevention of post-operative recurrence thereof, as well as methods for inducing CTLs, methods for inducing an immune response against tumor-associated endothelia and also anti-tumor immunity, which methods include the step of administering the IQGAP3 polypeptides, polynucleotides encoding IQGAP3 polypeptides, exosomes or the antigen-presenting cells presenting IQGAP3 polypeptides or the pharmaceutical agents of the invention. In addition, the CTLs of the invention also find use as vaccines against cancer.

The present invention can apply to any of diseases relating to IQGAP3 over-expression, such as cancer, including for example, bladder cancer, renal cancer, lung cancer, esophageal cancer, breast cancer, pancreatic cancer and gastric cancer. Preferred cancer targets include, but are not limited to gastric, lung, breast, bladder and pancreatic cancers.

In addition to the above, other objects and features of the invention will become more fully apparent when the following detailed description is read in conjunction with the accompanying figures and examples. However, it is to be understood that both the foregoing summary of the invention and the following detailed description are of exemplified embodiments, and not restrictive of the invention or other alternate embodiments of the invention. In particular, while the invention is described herein with reference to a number of specific embodiments, it will be appreciated that the description is illustrative of the invention and is not constructed as limiting of the invention. Various modifications and applications may occur to those who are skilled in the art, without departing from the spirit and the scope of the invention, as described by the appended claims. Likewise, other objects, features, benefits and advantages of the present invention will be apparent from this summary and certain embodiments described below, and will be readily apparent to those skilled in the art. Such objects, features, benefits and advantages will be apparent from the above in conjunction with the accompanying examples, data, figures and all reasonable inferences to be drawn therefrom, alone or with consideration of the references incorporated herein.

### BRIEF DESCRIPTION OF DRAWINGS

Various aspects and applications of the present invention will become apparent to the skilled artisan upon consideration of the brief description of the figures and the detailed description of the present invention and its preferred embodiments which follows.

FIGS. 1A and B include a series of photographs, (a)-(s), depicting the results of IFN-gamma ELISPOT assay on CTLs that were induced with peptides derived from IQGAP3. The CTLs in well numbers #3 and #6 stimulated with IQGAP3-A24-9-955 (SEQ ID NO:2) (a), #5 with IQGAP3-A24-9-1167 (SEQ ID NO:4) (b), #7 with IQGAP3-A24-9-779 (SEQ ID NO:7) (c), #2 with IQGAP3-A24-9-74 (SEQ ID NO: 21) (d), #8 with IQGAP3-A24-9-26 (SEQ ID NO:25) (e), #4 with IQGAP3-A24-9-137 (SEQ ID NO:29) (f), #8 with IQGAP3-A24-9-63 (SEQ ID NO:32) (g), #8 with IQGAP3-A24-10-1600 (SEQ ID NO:35) (h), #2 with IQGAP3-A24-10-1507 (SEQ ID NO:37) (i), #2 with IQGAP3-A24-10-139 (SEQ ID NO: 40) (j), #5 with IQGAP3-A24-10-1097 (SEQ ID NO:49) (k), #7 with IQGAP3-A24-10-345 (SEQ ID NO:53) (l), #1 with IQGAP3-A24-10-1614 (SEQ ID NO:55) (m), #3 with IQGAP3-A24-10-191 (SEQ ID NO:56) (n), #5 with IQGAP3-A24-10-314 (SEQ ID NO:57) (o), #5 with IQGAP3-A24-10-1363 (SEQ ID NO:62) (p), #7 with IQGAP3-A24-10-1114 (SEQ ID NO:63) (q) and #2 with IQGAP3-A24-10-1207 (SEQ ID NO: 67) (r) showed potent IFN-gamma production ability as compared with the control, respectively. In contrast, no specific IFN-gamma production was detected from the CTLs stimulated with IQGAP3-A24-9-417 (SEQ ID NO: 6) against peptide-pulsed target cells (s). The cells in the wells denoted with a rectangular box were expanded to establish CTL lines. In the figures, “+” indicates the IFN-gamma production against target cells pulsed with the appropriate peptide, and “−” indicates the IFN-gamma production against target cells not pulsed with any peptides. In the figure, “+” indicates that the cells in the wells were pulsed with appropriate peptides, and “−” indicates that the cells had not been pulsed with the peptides.

FIG. 1B is continuation of FIG. 1A.

FIGS. 2A, B, and C include a series of line graphs, (a)-(s), depicting the establishment of CTL lines stimulated with various IQGAP3 peptides, namely SEQ ID NO: 2 (a), SEQ ID NO: 4 (b), SEQ ID NO: 7 (c), SEQ ID NO: 21 (d), SEQ ID NO: 25 (e), SEQ ID NO: 29 (f), SEQ ID NO: 32 (g), SEQ ID NO: 35 (h), SEQ ID NO: 37 (i), SEQ ID NO: 40 (j), SEQ ID NO: 49 (k), SEQ ID NO: 53 (l), SEQ ID NO: 55 (m), SEQ ID NO: 56 (n), SEQ ID NO: 57 (O), SEQ ID NO: 62 (p), SEQ ID NO: 63 (q) and SEQ ID NO: 67 (r) with IFN-gamma ELISA assay. The results demonstrate that CTL lines established by stimulation with each peptide showed potent IFN-gamma production as compared with the control. In contrast, no specific IFN-gamma production was observed from the CTL line established with SEQ ID NO: 6 against peptide-pulsed target cells (s). In the figures, “+” indicates the IFN-gamma production against target cells pulsed with the appropriate peptides, and “−” indicates that the IFN-gamma production against target cells not pulsed with any peptides.

FIG. 2B is continuation of FIG. 2A.

FIG. 2C is continuation of FIG. 2B.

FIG. 3 is a line graph depicting the specific CTL activity against target cells that exogenously express IQGAP3 and HLA-A*2402. COS7 cells transfected with HLA-A*2402 or with the full length IQGAP3 gene were prepared as control. The CTL line established with IQGAP3-A24-9-779 (SEQ ID NO: 7) showed specific CTL activity against COS7 cells transfected with both IQGAP3 and HLA-A*2402 (black lozenge). In contrast, no significant specific CTL activity was detected against target cells expressing either HLA-A*2402 (triangle) or IQGAP3 (circle).

FIGS. 4A and B is composed of a series of photographs, (a)-(r), depicting the results of an IFN-gamma ELISPOT assay on CTLs that were induced with peptides derived from IQGAP3. The CTLs in the well number #6 and 6 stimulated with IQGAP3-A02-9-146 (SEQ ID NO: 75) (a), #6 with IQGAP3-A02-9-553 (SEQ ID NO: 85) (b), #1 with IQGAP3-A02-9-756 (SEQ ID NO: 101) (c), #7 with IQGAP3-A02-10-961 (SEQ ID NO: 111) (d), #7 and 6 with IQGAP3-A02-10-70 (SEQ ID NO: 114) (e), #5 with IQGAP3-A02-10-1174 (SEQ ID NO: 121) (f), #8 with IQGAP3-A02-10-548 (SEQ ID NO: 125) (g), #1 with IQGAP3-A02-10-903 (SEQ ID NO: 130) (h), #2 with IQGAP3-A02-10-953 (SEQ ID NO: 139) (i), #2 with IQGAP3-A02-10-1590 (SEQ ID NO: 140) (j), #2 with IQGAP3-A02-10-1424 (SEQ ID NO: 141) (k), #2 with IQGAP3-A02-10-416 (SEQ ID NO: 142) (l), #4 with IQGAP3-A02-10-67 (SEQ ID NO: 143) (m), #6 with IQGAP3-A02-10-1461 (SEQ ID NO: 145) (n), #5 with IQGAP3-A02-10-842 (SEQ ID NO: 148) (o), #3 with IQGAP3-A02-10-897 (SEQ ID NO: 150) (p) and #5 with IQGAP3-A02-9-1234 (SEQ ID NO: 99) (q) showed potent IFN-gamma production ability as compared with the control, respectively. In contrast, no specific IFN-gamma production was observed from the CTL stimulated with IQGAP3-A02-10-868 (SEQ ID NO: 113) against peptide-pulsed target cells (r). The cells in the wells denoted with a rectangular box were expanded to establish CTL lines. In the figures, “+” indicates the IFN-gamma production against target cells pulsed with the appropriate peptide, and “−” indicates the IFN-gamma production against target cells not pulsed with any peptides. In the figure, “+” indicates that the cells in the wells were pulsed with appropriate peptides, and “−” indicates that the cells had not been pulsed with the peptides.

FIG. 4B is continuation of FIG. 4A.

FIGS. 5A and B include a series of line graphs, (a)-(q), depicting the IFN-gamma production of CTL lines stimulated with various IQGAP3 peptides, namely IQGAP3-A02-9-146 (SEQ ID NO: 75) (a), IQGAP3-A02-9-553 (SEQ ID NO: 85) (b), IQGAP3-A02-9-756 (SEQ ID NO: 101) (c), IQGAP3-A02-10-961 (SEQ ID NO: 111) (d), IQGAP3-A02-10-70 (SEQ ID NO: 114) (e), IQGAP3-A02-10-1174 (SEQ ID NO: 121) (f), IQGAP3-A02-10-548 (SEQ ID NO: 125) (g), IQGAP3-A02-10-903 (SEQ ID NO: 130) (h), IQGAP3-A02-10-953 (SEQ ID NO: 139) (i), IQGAP3-A02-10-1590 (SEQ ID NO: 140) (j), IQGAP3-A02-10-1424 (SEQ ID NO: 141) (k), IQGAP3-A02-10-416 (SEQ ID NO: 142) (l), IQGAP3-A02-10-67 (SEQ ID NO: 143) (m), IQGAP3-A02-10-1461 (SEQ ID NO: 145) (n), IQGAP3-A02-10-842 (SEQ ID NO: 148) (o), IQGAP3-A02-10-897 (SEQ ID NO: 150) (p) and IQGAP3-A02-9-1234 (SEQ ID NO: 99) (q) detected by IFN-gamma ELISA assay. The results demonstrate that CTL lines stimulated with each peptide showed potent IFN-gamma production as compared with the control. In the figures, “+” indicates the IFN-gamma production against target cells pulsed with the appropriate peptides and “−” indicates that the IFN-gamma production against target cells not pulsed with any peptides.

FIG. 5B is continuation of FIG. 5A.

FIG. 5C is continuation of FIG. 5B.

FIG. 6 is composed of a series of line graphs, (a)-(f), depicting the IFN-gamma production of the CTL clones established by limiting dilution from the CTL lines stimulated with various IQGAP3 peptides, namely IQGAP3-A02-9-146 (SEQ ID NO: 75) (a), IQGAP3-A02-9-553 (SEQ ID NO: 85) (b), IQGAP3-A02-10-1174 (SEQ ID NO: 121) (c), IQGAP3-A02-10-903 (SEQ ID NO: 130) (d), IQGAP3-A02-10-67 (SEQ ID NO: 143) (e), and IQGAP3-A02-10-1461 (SEQ ID NO: 145) (f). The results demonstrate that the CTL clones established by stimulation with IQGAP3-A02-9-146 (SEQ ID NO: 75) (a), IQGAP3-A02-9-553 (SEQ ID NO: 85) (b), IQGAP3-A02-10-1174 (SEQ ID NO: 121) (c), IQGAP3-A02-10-903 (SEQ ID NO: 130) (d), IQGAP3-A02-10-67 (SEQ ID NO: 143) (e), and IQGAP3-A02-10-1461 (SEQ ID NO: 145) (f) showed potent IFN-gamma production as compared with the control. In the figure, “+” indicates the IFN-gamma production against target cells pulsed with IQGAP3-A02-9-146 (SEQ ID NO: 75) (a), IQGAP3-A02-9-553 (SEQ ID NO: 85) (b), IQGAP3-A02-10-1174 (SEQ ID NO: 121) (c), IQGAP3-A02-10-903 (SEQ ID NO: 130) (d), IQGAP3-A02-10-67 (SEQ ID NO: 143) (e), and IQGAP3-A02-10-1461 (SEQ ID NO: 145) (f) and “−” indicates the IFN-gamma production against target cells not pulsed with any peptides.

FIG. 7 is a line graph depicting the specific CTL activity against the target cells that exogenously express IQGAP3 and HLA-A*0201. COS7 cells transfected with HLA-A*0201 or with the full length IQGAP3 gene were prepared as controls. The CTL clone established with IQGAP3-A02-9-553 (SEQ ID NO: 85) (a) and IQGAP3-A02-9-1234 (SEQ ID NO: 99) (b) showed specific CTL activity against COS7 cells transfected with both IQGAP3 and HLA-A*0201 (black lozenge). On the other hand, no significant specific CTL activity was detected against target cells expressing either HLA-A*0201 (triangle) or IQGAP3 (circle).

### DESCRIPTION OF EMBODIMENTS

Although any methods and materials similar or equivalent to those described herein can be used in the practice or testing of embodiments of the present invention, the preferred methods, devices, and materials are now described. However, before the present materials and methods are described, it is to be understood that the present invention is not limited to the particular sizes, shapes, dimensions, materials, methodologies, protocols, etc. described herein, as these may vary in accordance with routine experimentation and optimization. It is also to be understood that the terminology used in the description is for the purpose of describing the particular versions or embodiments only, and is not intended to limit the scope of the present invention which will be limited only by the appended claims.

The disclosure of each publication, patent or patent application mentioned in this specification is specifically incorporated by reference herein in its entirety. However, nothing herein is to be construed as an admission that the invention is not entitled to antedate such disclosure by virtue of prior invention.

### I. DEFINITIONS

Unless otherwise defined, all technical and scientific terms used herein have the same meaning as commonly understood by one of ordinary skill in the art to which the present invention belongs. However, in case of conflict, the present specification, including definitions, will control.

The words “a”, “an”, and “the” as used herein mean “at least one” unless otherwise specifically indicated.

The terms “polypeptide”, “peptide” and “protein” are used interchangeably herein to refer to a polymer of amino acid residues. The terms apply to amino acid polymers in which one or more amino acid residue is a modified residue, or a non-naturally occurring residue, such as an artificial chemical mimetic of a corresponding naturally occurring amino acid, as well as to naturally occurring amino acid polymers.

The term “amino acid” as used herein refers to naturally occurring and synthetic amino acids, as well as amino acid analogs and amino acid mimetics that similarly function to the naturally occurring amino acids. Naturally occurring amino acids are those encoded by the genetic code, as well as those modified after translation in cells (e.g., hydroxyproline, gamma-carboxyglutamate, and O-phosphoserine). The phrase “amino acid analog” refers to compounds that have the same basic chemical structure (an alpha carbon bound to a hydrogen, a carboxy group, an amino group, and an R group) as a naturally occurring amino acid but have a modified R group or modified backbones (e.g., homoserine, norleucine, methionine, sulfoxide, methionine methyl sulfonium). The phrase “amino acid mimetic” refers to chemical compounds that have different structures but similar functions to general amino acids.

Amino acids may be referred to herein by their commonly known three letter symbols or the one-letter symbols recommended by the IUPAC-IUB Biochemical Nomenclature Commission.

The terms “gene”, “polynucleotides”, “nucleotides” and “nucleic acids” are used interchangeably herein and, unless otherwise specifically indicated, are referred to by their commonly accepted single-letter codes.

Unless otherwise defined, the terms “cancer” refers to the cancers over-expressing the IQGAP3 gene, examples of which include, but are not limited to, bladder cancer, renal cancer, lung cancer, esophageal cancer, gastric cancer, breast cancer, and pancreatic cancer.

Unless otherwise defined, the term “cytotoxic T lymphocyte”, “cytotoxic T cell” and “CTL” are used interchangeably herein and, otherwise specifically indicated, refer to a sub-group of T lymphocytes that are capable of recognizing non-self cells (e.g., tumor cells, virus-infected cells) and inducing the death of such cells.

### II. PEPTIDES

To demonstrate that peptides derived from IQGAP3 function as an antigen recognized by cytotoxic T lymphocytes (CTLs), peptides derived from IQGAP3 (SEQ ID NO: 154) were analyzed to determine whether they were antigen epitopes restricted by HLA-A24 or HLA-A02, which are commonly encountered HLA alleles (Date Y et al., Tissue Antigens 47: 93-101, 1996; Kondo A et al., J Immunol 155: 4307-12, 1995; Kubo R T et al., J Immunol 152: 3913-24, 1994). Candidates of HLA-A24 and HLA-A02 binding peptides derived from IQGAP3 were identified based on their binding affinities to HLA-A24 and HLA-A02. After in vitro stimulation of T-cells by dendritic cells (DCs) loaded with these peptides, CTLs were successfully established using each of the following peptides.

| | |
|---|---|
| IQGAP3-A24-9-955, | (SEQ ID NO: 2) |
| IQGAP3-A24-9-1167, | (SEQ ID NO: 4) |
| IQGAP3-A24-9-779, | (SEQ ID NO: 7) |
| IQGAP3-A24-9-74, | (SEQ ID NO: 21) |
| IQGAP3-A24-9-26, | (SEQ ID NO: 25) |
| IQGAP3-A24-9-137, | (SEQ ID NO: 29) |
| IQGAP3-A24-9-63, | (SEQ ID NO: 32) |
| IQGAP3-A24-10-1600, | (SEQ ID NO: 35) |
| IQGAP3-A24-10-1507, | (SEQ ID NO: 37) |
| IQGAP3-A24-10-139, | (SEQ ID NO: 40) |
| IQGAP3-A24-10-1097, | (SEQ ID NO: 49) |
| IQGAP3-A24-10-345, | (SEQ ID NO: 53) |
| IQGAP3-A24-10-1614, | (SEQ ID NO: 55) |
| IQGAP3-A24-10-191, | (SEQ ID NO: 56) |
| IQGAP3-A24-10-314, | (SEQ ID NO: 57) |
| IQGAP3-A24-10-1363, | (SEQ ID NO: 62) |
| IQGAP3-A24-10-1114, | (SEQ ID NO: 63) |
| IQGAP3-A24-10-1207, | (SEQ ID NO: 67) |
| IQGAP3-A02-9-146, | (SEQ ID NO: 75) |
| IQGAP3-A02-9-553, | (SEQ ID NO: 85) |
| IQGAP3-A02-9-1234, | (SEQ ID NO: 99) |
| IQGAP3-A02-9-756, | (SEQ ID NO: 101) |
| IQGAP3-A02-10-961, | (SEQ ID NO: 111) |
| IQGAP3-A02-10-70, | (SEQ ID NO: 114) |
| IQGAP3-A02-10-1174, | (SEQ ID NO: 121) |
| IQGAP3-A02-10-548, | (SEQ ID NO: 125) |
| IQGAP3-A02-10-903, | (SEQ ID NO: 130) |
| IQGAP3-A02-10-953, | (SEQ ID NO: 139) |
| IQGAP3-A02-10-1590, | (SEQ ID NO: 140) |
| IQGAP3-A02-10-1424, | (SEQ ID NO: 141) |
| IQGAP3-A02-10-416, | (SEQ ID NO: 142) |
| IQGAP3-A02-10-67, | (SEQ ID NO: 143) |
| IQGAP3-A02-10-1461, | (SEQ ID NO: 145) |
| IQGAP3-A02-10-842 | (SEQ ID NO: 148) |
| and | |
| IQGAP3-A02-10-897. | (SEQ ID NO: 150) |

These established CTLs show potent specific CTL activity against target cells pulsed with respective peptides. These results herein demonstrate that IQGAP3 is an antigen recognized by CTL and that the peptides may be epitope peptides of IQGAP3 restricted by HLA-A24 or HLA-A02.

Since the IQGAP3 gene is over expressed in most cancer tissues, such as gastric, renal, esophageal, lung, breast, bladder and pancreatic cancer, it represents a good target for immunotherapy. Thus, the present invention provides nonapeptides (peptides consisting of nine amino acid residues) and decapeptides (peptides consisting of ten amino acid residues) corresponding to CTL-recognized epitopes of IQGAP3. Particularly preferred examples of nonapeptides and decapeptides of the present invention include those peptides consisting of the amino acid sequence selected from among SEQ ID NOs: 2, 4, 7, 21, 25, 29, 32, 35, 37, 40, 49, 53, 55, 56, 57, 62, 63, 67, 75, 85, 99, 101, 111, 114, 121, 125, 130, 139, 140, 141, 142, 143, 145, 148 and 150.

Generally, software programs presently available on the Internet, such as those described in Parker K C et al., J Immunol 1994 Jan. 1, 152(1): 163-75, can be used to calculate the binding affinities between various peptides and HLA antigens in silico. Binding affinity with HLA antigens can be measured as described, for example, in Parker K C et al., J Immunol 1994 Jan. 1, 152(1): 163-75; and Kuzushima K et al., Blood 2001, 98(6): 1872-81. The methods for determining binding affinity is described, for example, in the Journal of Immunological Methods, 1995, 185: 181-190 and Protein Science, 2000, 9: 1838-1846. Thus, the present invention encompasses peptides of IQGAP3 which bind with HLA antigens identified using such known programs.

The nonapeptides and decapeptides of the present invention can be flanked with additional amino acid residues so long as the resulting peptide retains its CTL inducibility. Such peptides having CTL inducibility are typically less than about 40 amino acids, often less than about 20 amino acids, usually less than about 15 amino acids. The particular amino acid sequences flanking the nonapeptides and decapeptides of the present invention (e.g., peptides consisting of the amino acid sequence selected from among SEQ ID NOs: 2, 4, 7, 21, 25, 29, 32, 35, 37, 40, 49, 53, 55, 56, 57, 62, 63, 67, 75, 85, 99, 101, 111, 114, 121, 125, 130, 139, 140, 141, 142, 143, 145, 148 or 150) is not limited and can be composed of any kind of amino acids so long as it does not impair the CTL inducibility of the original peptide. Thus, the present invention also provides peptides having CTL inducibility and an amino acid sequence selected from among SEQ ID NOs: 2, 4, 7, 21, 25, 29, 32, 35, 37, 40, 49, 53, 55, 56, 57, 62, 63, 67, 75, 85, 99, 101, 111, 114, 121, 125, 130, 139, 140, 141, 142, 143, 145, 148 or 150.

In general, the modification of one or more amino acids in a protein will not influence the function of the protein, and in some cases will even enhance the desired function of the original protein. In fact, modified peptides (i.e., peptides composed of an amino acid sequence in which one, two or several amino acid residues have been modified (i.e., substituted, added or inserted) as compared to an original reference sequence) have been known to retain the biological activity of the original peptide (Mark et al., Proc Natl Acad Sci USA 1984, 81: 5662-6; Zoller and Smith, Nucleic Acids Res 1982, 10: 6487-500; Dalbadie-McFarland et al., Proc Natl Acad Sci USA 1982, 79: 6409-13). Thus, in one embodiment, the peptides of the present invention may have both CTL inducibility and an amino acid sequence selected from among SEQ ID NOs: 2, 4, 7, 21, 25, 29, 32, 35, 37, 40, 49, 53, 55, 56, 57, 62, 63, 67, 75, 85, 99, 101, 111, 114, 121, 125, 130, 139, 140, 141, 142, 143, 145, 148 and 150, wherein one, two or even more amino acids are inserted, added and/or substituted.

Those of skill in the art recognize that individual additions or substitutions to an amino acid sequence which alter a single amino acid or a small percentage of amino acids tend to result in the conservation of the properties of the original amino acid side-chain. As such, they are often referred to as “conservative substitutions” or “conservative modifications”, wherein the alteration of a protein results in a modified protein having a function analogous to the original protein. Conservative substitution tables providing functionally similar amino acids are well known in the art. Examples of amino acid side chain characteristics that are desirable to conserve include, for example, hydrophobic amino acids (A, I, L, M, F, P, W, Y, V), hydrophilic amino acids (R, D, N, C, E, Q, G, H, K, S, T), and side chains having the following functional groups or characteristics in common: an aliphatic side-chain (G, A, V, L, I, P); a hydroxyl group containing side-chain (S, T, Y); a sulfur atom containing side-chain (C, M); a carboxylic acid and amide containing side-chain (D, N, E, Q); a base containing side-chain (R, K, H); and an aromatic containing side-chain (H, F, Y, W). In addition, the following eight groups each contain amino acids that are accepted in the art as conservative substitutions for one another:

### 1) Alanine (A), Glycine (G);

2) Aspartic acid (D), Glutamic acid (E);

### 3) Aspargine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

8) Cysteine (C), Methionine (M) (see, e.g., Creighton, Proteins 1984).

Such conservatively modified peptides are also considered to be peptides of the present invention. However, peptides of the present invention are not restricted thereto and can include non-conservative modifications, so long as the modified peptide retains the CTL inducibility of the original peptide. Furthermore, modified peptides should not exclude CTL inducible peptides of polymorphic variants, interspecies homologues, and alleles of IQGAP3.

To retain the requisite CTL inducibility one can modify (insert, add and/or substitute) a small number (for example, 1, 2 or several) or a small percentage of amino acids. Herein, the term “several” means 5 or fewer amino acids, for example, 4 or 3 or fewer. The percentage of amino acids to be modified is preferably 20% or less, more preferably 15% or less, even more preferably 10% or less or 1 to 5%.

Homology analysis of preferred peptides of the present invention, IQGAP3-A24-9-955 (SEQ ID NO:2), IQGAP3-A24-9-1167 (SEQ ID NO:4), IQGAP3-A24-9-779 (SEQ ID NO:7), IQGAP3-A24-9-74 (SEQ ID NO: 21), IQGAP3-A24-9-26 (SEQ ID NO:25), IQGAP3-A24-9-137 (SEQ ID NO:29), IQGAP3-A24-9-63 (SEQ ID NO:32), IQGAP3-A24-10-1600 (SEQ ID NO:35), IQGAP3-A24-10-1507 (SEQ ID NO:37), IQGAP3-A24-10-139 (SEQ ID NO: 40), IQGAP3-A24-10-1097 (SEQ ID NO:49), IQGAP3-A24-10-345 (SEQ ID NO:53), IQGAP3-A24-10-1614 (SEQ ID NO:55), IQGAP3-A24-10-191 (SEQ ID NO:56), IQGAP3-A24-10-314 (SEQ ID NO:57), IQGAP3-A24-10-1363 (SEQ ID NO:62), IQGAP3-A24-10-1114 (SEQ ID NO:63), IQGAP3-A24-10-1207 (SEQ ID NO: 67), IQGAP3-A02-9-146 (SEQ ID NO:75), IQGAP3-A02-9-553 (SEQ ID NO:85), IQGAP3-A02-9-1234 (SEQ ID NO: 99), IQGAP3-A02-9-756 (SEQ ID NO:101), IQGAP3-A02-10-961 (SEQ ID NO:111), IQGAP3-A02-10-70 (SEQ ID NO:114), IQGAP3-A02-10-1174 (SEQ ID NO:121), IQGAP3-A02-10-548 (SEQ ID NO:125), IQGAP3-A02-10-903 (SEQ ID NO:130), IQGAP3-A02-10-953 (SEQ ID NO:139), IQGAP3-A02-10-1590 (SEQ ID NO:140), IQGAP3-A02-10-1424 (SEQ ID NO:141), IQGAP3-A02-10-416 (SEQ ID NO:142), IQGAP3-A02-10-67 (SEQ ID NO:143), IQGAP3-A02-10-1461 (SEQ ID NO:145), IQGAP3-A02-10-842 (SEQ ID NO:148) and IQGAP3-A02-10-897 (SEQ ID NO:150) confirmed that these peptides do not have significant homology with peptides derived from any other known human gene products. Thus, the possibility of these peptides generating unknown or undesired immune responses when used for immunotherapy is significantly lowered. Accordingly, these peptides are expected to be highly useful for eliciting immunity in tumor patients against IQGAP3 on cancer cells, such as renal, esophageal, gastric, lung, breast, bladder and pancreatic cancer.

When used in the context of immunotherapy, peptides of the present invention should be presented on the surface of a cell or exosome, preferably as a complex with an HLA antigen. Therefore, it is preferable to select peptides that not only induce CTLs but also possess high binding affinity to the HLA antigen. To that end, the peptides can be modified by substitution, insertion, deletion and/or addition of the amino acid residues to yield a modified peptide having improved binding affinity. In addition to peptides that are naturally displayed, since the regularity of the sequences of peptides displayed by binding to HLA antigens is already known (J Immunol 1994, 152: 3913; Immunogenetics 1995, 41: 178; J Immunol 1994, 155: 4307), modifications based on such regularity can be introduced into the immunogenic peptides of the invention. For example, it may be desirable to substitute the second amino acid from the N-terminus with phenylalanine, tyrosine, methionine, or tryptophan, and/or the amino acid at the C-terminus with phenylalanine, leucine, isoleucine, tryptophan, or methionine in order to increase the HLA-A24 binding. Thus, peptides having the amino acid sequences of SEQ ID NOs: 2, 4, 7, 21, 25, 29, 32, 35, 37, 40, 49, 53, 55, 56, 57, 62, 63 67 wherein the second amino acid from the N-terminus of the amino acid sequence of said SEQ ID NOs is substituted with phenylalanine, tyrosine, methionine, or tryptophan, and/or wherein the C-terminus of the amino acid sequence of said SEQ ID NOs is substituted with phenylalanine, leucine, isoleucine, tryptophan, or methionine are encompassed by the present invention. On the other hand, peptides possessing high HLA-A02 binding affinity have their second amino acid from the N-terminus is substituted with leucine or methionine, and peptides whose amino acid at C-terminus is substituted with valine or leucine. Thus, peptides having the amino acid sequences of SEQ ID NOs: 75, 85, 99, 101, 111, 114, 121, 125, 130, 139, 140, 141, 142, 143, 145, 148 or 150, wherein the second amino acid from the N-terminus of the amino acid sequence of said SEQ ID NOs is substituted with leucine or methionine, and peptides, and/or wherein the C-terminus of the amino acid sequence of said SEQ ID NOs is substituted with valine or leucine are encompassed by the present invention. Substitutions can be introduced not only at the terminal amino acids but also at the position of potential TCR recognition of peptides. Several studies have demonstrated that amino acid substitutions in a peptide can be equal to or better than the original, for example CAP1, p53₍₂₆₄₋₂₇₂₎, Her-2/neu₍₃₆₉₋₃₇₇₎ or gp100₍₂₀₉₋₂₁₇₎ (Zaremba et al. Cancer Res. 57, 4570-4577, 1997, T. K. Hoffmann et al. J. Immunol. (2002) Feb. 1; 168(3):1338-47., S. O. Dionne et al. Cancer Immunol immunother. (2003) 52: 199-206 and S. O. Dionne et al. Cancer Immunology, Immunotherapy (2004) 53, 307-314).

The present invention also contemplates the addition of one to two amino acids to the N and/or C-terminus of the described peptides. Such modified peptides having high HLA antigen binding affinity and retained CTL inducibility are also included in the present invention.

However, when the peptide sequence is identical to a portion of the amino acid sequence of an endogenous or exogenous protein having a different function, side effects such as autoimmune disorders and/or allergic symptoms against specific substances may be induced. Therefore, it is preferable to first perform homology searches using available databases to avoid situations in which the sequence of the peptide matches the amino acid sequence of another protein. When it becomes clear from the homology searches that there exists not even a peptide with 1 or 2 amino acid differences as compared to the objective peptide, the objective peptide can be modified in order to increase its binding affinity with HLA antigens, and/or increase its CTL inducibility without any danger of such side effects.

Although peptides having high binding affinity to the HLA antigens as described above are expected to be highly effective, the candidate peptides, which are selected according to the presence of high binding affinity as an indicator, are further examined for the presence of CTL inducibility. Herein, the phrase “CTL inducibility” indicates the ability of the peptide to induce cytotoxic lymphocytes (CTLs) when presented on antigen-presenting cells. Further, “CTL inducibility” includes the ability of the peptide to induce CTL activation, CTL proliferation, promote CTL lysis of target cells, and to increase CTL IFN-gamma production.

Confirmation of CTL inducibility is accomplished by inducing antigen-presenting cells carrying human MHC antigens (for example, B-lymphocytes, macrophages, and dendritic cells (DCs)), or more specifically DCs derived from human peripheral blood mononuclear leukocytes, and after stimulation with the peptides, mixing with CD8-positive cells, and then measuring the IFN-gamma (IFN-gamma) produced and released by CTL against the target cells. As the reaction system, transgenic animals that have been produced to express a human HLA antigen (for example, those described in BenMohamed L, Krishnan R, Longmate J, Auge C, Low L, Primus J, Diamond D J, Hum Immunol 2000 August, 61(8): 764-79, Related Articles, Books, Linkout Induction of CTL response by a minimal epitope vaccine in HLA A*0201/DR1 transgenic mice: dependence on HLA class II restricted T(H) response) can be used. For example, the target cells can be radiolabeled with ⁵¹Cr and such, and cytotoxic activity can be calculated from radioactivity released from the target cells. Alternatively, CTL inducibility can be assessed by measuring IFN-gamma (IFN-gamma) produced and released by CTL in the presence of antigen-presenting cells (APCs) that carry immobilized peptides, and visualizing the inhibition zone on the media using anti-IFN-gamma monoclonal antibodies.

As a result of examining the CTL inducibility of the peptides as described above, it was discovered that those peptides having high binding affinity to an HLA antigen did not necessarily have high inducibility. However, of those peptides identified and assessed, nonapeptides or decapeptides having the amino acid sequences of SEQ ID NOs: 2, 4, 7, 21, 25, 29, 32, 35, 37, 40, 49, 53, 55, 56, 57, 62, 63, 67, 75, 85, 99, 101, 111, 114, 121, 125, 130, 139, 140, 141, 142, 143, 145, 148 and 150, were found to exhibit particularly high CTL inducibility as well as high binding affinity to an HLA antigen. Thus, these peptides are exemplified as preferred embodiments of the present invention.

In addition to the above-described modifications, the peptides of the present invention can also be linked to other substances, so long as the resulting linked peptide retains the requisite CTL inducibility of the original peptide. Examples of suitable substances include, but are not limited to: peptides, lipids, sugar and sugar chains, acetyl groups, natural and synthetic polymers, etc. The peptides can contain modifications such as glycosylation, side chain oxidation, or phosphorylation, etc., provided the modifications do not destroy the biological activity of the original peptide. These kinds of modifications can be performed to confer additional functions (e.g., targeting function, and delivery function) or to stabilize the polypeptide.

For example, to increase the in vivo stability of a polypeptide, it is known in the art to introduce D-amino acids, amino acid mimetics or unnatural amino acids; this concept can also be adapted to the present polypeptides. The stability of a polypeptide can be assayed in a number of ways. For instance, peptidases and various biological media, such as human plasma and serum, can be used to test stability (see, e.g., Verhoef et al., Eur J Drug Metab Pharmacokin 1986, 11: 291-302).

Herein, the peptides of the present invention can also be described as “IQGAP3 peptide(s)” or “IQGAP3 polypeptide(s)”.

The peptides of the present invention are presented on the surface of a cell (e.g. antigen presenting cell) or an exosome as complexes in combination with HLA antigens and then induce CTLs. Therefore, the peptides formed complexes with HLA antigens on the surface of a cells or an exsosomes are also included in the present invention. Such exosomes can be prepared, for example using the methods detailed in Japanese Patent Application Kohyo Publications Nos. Hei 11-510507 and WO99/03499, and can be prepared using APCs obtained from patients who are subject to treatment and/or prevention. The exosomes or cells presenting the peptides of the present invention can be inoculated as vaccines.

The type of HLA antigens contained in the above complexes must match that of the subject requiring treatment and/or prevention. For example, in the Japanese population, HLA-A24 and HLA-A02 is prevalent and therefore would be appropriate for treatment of a Japanese patient. The use of the A24 and A02 type that is highly expressed among the Japanese and Caucasian is favorable for obtaining effective results, and subtypes also find use. Typically, in the clinic, the type of HLA antigen of the patient requiring treatment is investigated in advance, which enables the appropriate selection of peptides having high levels of binding affinity to the particular antigen, or having CTL inducibility by antigen presentation.

When using the A24 and A02 type HLA antigen for the exosome or cell, the peptides having the amino acid sequence selected among from SEQ ID NO: 2, 4, 7, 21, 25, 29, 32, 35, 37, 40, 49, 53, 55, 56, 57, 62, 63, 67, 75, 85, 99, 101, 111, 114, 121, 125, 130, 139, 140, 141, 142, 143, 145, 148 and 150 are preferably used.

### III. PREPARATION OF IQGAP3 PEPTIDES

The peptides of the invention can be prepared using well known techniques. For example, the peptides can be prepared synthetically, using recombinant DNA technology or chemical synthesis. Peptide of the invention can be synthesized individually or as longer polypeptides composed of two or more peptides. The peptides can then be isolated i.e., purified, so as to be substantially free of other naturally occurring host cell proteins and fragments thereof, or any other chemical substances.

A peptide of the present invention can be obtained through chemical synthesis based on the selected amino acid sequence. Examples of conventional peptide synthesis methods that can be adapted to the synthesis include, but are not limited to:

(i) Peptide Synthesis, Interscience, New York, 1966;

(ii) The Proteins, Vol. 2, Academic Press, New York, 1976;

(iii) Peptide Synthesis (in Japanese), Maruzen Co., 1975;

(iv) Basics and Experiment of Peptide Synthesis (in Japanese), Maruzen Co., 1985;

(v) Development of Pharmaceuticals (second volume) (in Japanese), Vol. 14 (peptide synthesis), Hirokawa, 1991;

(vi) WO99/67288; and

(vii) Barany G. & Merrifield R. B., Peptides Vol. 2, “Solid Phase Peptide Synthesis”, Academic Press, New York, 1980, 100-118.

Alternatively, the present peptides can be obtained adapting any known genetic engineering methods for producing peptides (e.g., Morrison J, J Bacteriology 1977, 132: 349-51; Clark-Curtiss & Curtiss, Methods in Enzymology (eds. Wu et al.) 1983, 101: 347-62). For example, first, a suitable vector harboring a polynucleotide encoding the objective peptide in an expressible form (e.g., downstream of a regulatory sequence corresponding to a promoter sequence) is prepared and transformed into a suitable host cell. The host cell is then cultured to produce the peptide of interest. The peptide can also be produced in vitro adopting an in vitro translation system.

### IV. POLYNUCLEOTIDES

The present invention also provides a polynucleotide which encodes any of the aforementioned peptides of the present invention. These include polynucleotides derived from the natural occurring IQGAP3 gene (GenBank Accession No. NM_{—}178229 (SEQ ID NO: 153)) as well as those having a conservatively modified nucleotide sequence thereof. Herein, the phrase “conservatively modified nucleotide sequence” refers to sequences which encode identical or essentially identical amino acid sequences. Due to the degeneracy of the genetic code, a large number of functionally identical nucleic acids encode any given protein. For instance, the codons GCA, GCC, GCG, and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a peptide also describes every possible silent variation of the nucleic acid. One of ordinary skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a peptide is implicitly described in each disclosed sequence.

The polynucleotide of the present invention can be composed of DNA, RNA, and derivatives thereof. A DNA is suitably composed of bases such as A, T, C, and G, and T is replaced by U in an RNA.

The polynucleotide of the present invention can encode multiple peptides of the present invention, with or without intervening amino acid sequences in between. For example, the intervening amino acid sequence can provide a cleavage site (e.g., enzyme recognition sequence) of the polynucleotide or the translated peptides. Furthermore, the polynucleotide can include any additional sequences to the coding sequence encoding the peptide of the present invention. For example, the polynucleotide can be a recombinant polynucleotide that includes regulatory sequences required for the expression of the peptide or can be an expression vector (plasmid) with marker genes and such. In general, such recombinant polynucleotides can be prepared by the manipulation of polynucleotides through conventional recombinant techniques using, for example, polymerases and endonucleases.

Both recombinant and chemical synthesis techniques can be used to produce the polynucleotides of the present invention. For example, a polynucleotide can be produced by insertion into an appropriate vector, which can be expressed when transfected into a competent cell. Alternatively, a polynucleotide can be amplified using PCR techniques or expression in suitable hosts (see, e.g., Sambrook et al., Molecular Cloning: A Laboratory Manual, Cold Spring Harbor Laboratory, New York, 1989). Alternatively, a polynucleotide can be synthesized using the solid phase techniques, as described in Beaucage S L & Iyer R P, Tetrahedron 1992, 48: 2223-311; Matthes et al., EMBO J. 1984, 3: 801-5.

### V. ANTIGEN-PRESENTING CELLS (APCs)

The present invention also provides antigen-presenting cells (APCs) that present complexes formed between HLA antigens and the peptides of the present invention on its surface. The APCs that are obtained by contacting the peptides of the present invention, or introducing the nucleotides encoding the peptides of the present invention in an expressible form can be derived from patients who are subject to treatment and/or prevention, and can be administered as vaccines by themselves or in combination with other drugs including the peptides of the present invention, exosomes, or cytotoxic T cells.

The APCs are not limited to a particular kind of cells and include dendritic cells (DCs), Langerhans cells, macrophages, B cells, and activated T cells, which are known to present proteinaceous antigens on their cell surface so as to be recognized by lymphocytes. Since DC is a representative APC having the strongest CTL inducing action among APCs, DCs find use as the APCs of the present invention.

For example, an APC can be obtained by inducing DCs from peripheral blood monocytes and then contacting (stimulating) them with the peptides of the present invention in vitro, ex vivo or in vivo. When the peptides of this invention are administered to the subjects, APCs that present the peptides of the present invention are induced in the body of the subject. The phrase “inducing APC” includes contacting (stimulating) a cell with the peptides of the present invention, or nucleotides encoding the peptides of the present invention to present complexes formed between HLA antigens and the peptides of the present invention on cell's surface. Alternatively, after introducing the peptides of the present invention to the APCs to allow the APCs to present the peptides, the APCs can be administered to the subject as a vaccine. For example, the ex vivo administration can include the steps of:

a: collecting APCs from a first subject:,

b: contacting with the APCs of step a, with the peptide and

c: administering the peptide-loaded APCs to a second subject.

The first subject and the second subject can be the same individual, or may be different individuals. Alternatively, according to the present invention, use of the peptides of the present invention for manufacturing a pharmaceutical composition inducing antigen-presenting cells is provided. In addition, the present invention provides a method or process for manufacturing a pharmaceutical composition inducing antigen-presenting cells. Further, the present invention also provides the peptides of the present invention for inducing antigen-presenting cells. The APCs obtained by step (b) can be administered to the subject as a vaccine.

According to an aspect of the present invention, the APCs have a high level of CTL inducibility. In the term of “high level of CTL inducibility”, the high level is relative to the level of that by APC contacting with no peptide or peptides which can not induce the CTL. Such APCs having a high level of CTL inducibility can be prepared by a method which includes the step of transferring genes containing polynucleotides that encode the peptides of the present invention to APCs in vitro. The introduced genes can be in the form of DNAs or RNAs. Examples of methods for introduction include, without particular limitations, various methods conventionally performed in this field, such as lipofection, electroporation, and calcium phosphate method can be used. More specifically, it can be performed as described in Cancer Res 1996, 56: 5672-7; J Immunol 1998, 161: 5607-13; J Exp Med 1996, 184: 465-72; Published Japanese Translation of International Publication No. 2000-509281. By transferring the gene into APCs, the gene undergoes transcription, translation, and such in the cell, and then the obtained protein is processed by MHC Class I or Class II, and proceeds through a presentation pathway to present peptides.

### VI. CYTOTOXIC T CELLS

A cytotoxic T cell induced against any of the peptides of the present invention strengthens the immune response targeting tumor-associated endothelia in vivo and thus can be used as vaccines, in a fashion similar to the peptides per se. Thus, the present invention also provides isolated cytotoxic T cells that are specifically induced or activated by any of the present peptides.

Such cytotoxic T cells can be obtained by (1) administering to a subject or (2) contacting (stimulating) subject-derived APCs, and CD8-positive cells, or peripheral blood mononuclear leukocytes in vitro with the peptides of the present invention.

The cytotoxic T cells, which have been induced by stimulation from APCs that present the peptides of the present invention, can be derived from patients who are subject to treatment and/or prevention, and can be administered by themselves or in combination with other drugs including the peptides of this invention or exosomes for the purpose of regulating effects. The obtained cytotoxic T cells act specifically against target cells presenting the peptides of the present invention, or for example, the same peptides used for induction. The target cells can be cells that endogenously express IQGAP3, or cells that are transfected with the IQGAP3 gene; and cells that present a peptide of the present invention on the cell surface due to stimulation by the peptide can also serve as targets of activated CTL attack.

### VII. T Cell Receptor (TCR)

The present invention also provides a composition containing nucleic acids sequence encoding polypeptides that are capable of forming a subunit of a T cell receptor (TCR), and methods of using the same. The TCR subunits have the ability to form TCRs that confer specificity to T cells against tumor cells presenting IQGAP3. By using the known methods in the art, the nucleic acids sequence of alpha- and beta-chains comprising of the TCR expressing in the CTL induced with one or more peptides of the present invention can be identified (WO2007/032255 and Morgan et al., J Immunol, 171, 3288 (2003)). The derivative TCRs can bind target cells displaying the IQGAP3 peptide with high avidity, and optionally mediate efficient killing of target cells presenting the IQGAP3 peptide in vivo and in vitro.

The nucleic acids sequence encoding the TCR subunits can be incorporated into suitable vectors e.g. retroviral vectors. These vectors are well known in the art. The nucleic acids or the vectors containing them usefully can be transferred into a T cell, for example, a T cell from a patient. Advantageously, the invention provides an off-the-shelf composition allowing rapid modification of a patient's own T cells (or those of another mammal) to rapidly and easily produce modified T cells having excellent cancer cell killing properties.

Also, the present invention provides CTLs which are prepared by transduction with the nucleic acids encoding the TCR subunits polypeptides that bind to the IQGAP3 peptide e.g. SEQ ID NOs: 2, 4, 7, 21, 25, 29, 32, 35, 37, 40, 49, 53, 55, 56, 57, 62, 63, 67, 75, 85, 99, 101, 111, 114, 121, 125, 130, 139, 140, 141, 142, 143, 145, 148 and 150 in the context of HLA-A24 or HLA-A02. The transduced CTLs are capable of homing to cancer cells in vivo, and can be expanded by well known culturing methods in vitro (e.g., Kawakami et al., J. Immunol., 142, 3452-3461 (1989)). The T cells of the invention can be used to form an immunogenic composition useful in treating or the prevention of cancer in a patient in need of therapy or protection (WO2006/031221).

Prevention and prophylaxis include any activity which reduces the burden of mortality or morbidity from disease. Prevention and prophylaxis can occur “at primary, secondary and tertiary prevention levels.” While primary prevention and prophylaxis avoid the development of a disease, secondary and tertiary levels of prevention and prophylaxis encompass activities aimed at the prevention and prophylaxis of the progression of a disease and the emergence of symptoms as well as reducing the negative impact of an already established disease by restoring function and reducing disease-related complications. Alternatively, prevention and prophylaxis include a wide range of prophylactic therapies aimed at alleviating the severity of the particular disorder, e.g. reducing the proliferation and metastasis of tumors.

Treating and/or for the prophylaxis of cancer or, and/or the prevention of post-operative recurrence thereof includes any of the following steps, such as surgical removal of cancer cells, inhibition of the growth of cancerous cells, involution or regression of a tumor, induction of remission and suppression of occurrence of cancer, tumor regression, and reduction or inhibition of metastasis. Effectively treating and/or the prophylaxis of cancer decreases mortality and improves the prognosis of individuals having cancer, decreases the levels of tumor markers in the blood, and alleviates detectable symptoms accompanying cancer. For example, reduction or improvement of symptoms constitutes effectively treating and/or the prophylaxis include 10%, 20%, 30% or more reduction, or stable disease.

### VIII. PHARMACEUTICAL AGENTS OR COMPOSITIONS

Since IQGAP3 expression is specifically elevated in gastric cancer as compared with normal tissue (Jinawath N et al., AACR 2006), the peptides of the present invention or polynucleotides encoding such peptides can be used for the treatment and/or prophylaxis of cancer, and/or prevention of postoperative recurrence thereof. Thus, the present invention provides a pharmaceutical agent or composition for the treatment and/or for the prophylaxis of cancer, and/or prevention of postoperative recurrence thereof, which includes one or more of the peptides of the present invention, or polynucleotides encoding the peptides as an active ingredient. Alternatively, the present peptides can be expressed on the surface of any of the foregoing exosomes or cells, such as APCs for the use as pharmaceutical agents or compositions. In addition, the aforementioned cytotoxic T cells which target any of the peptides of the invention can also be used as the active ingredient of the present pharmaceutical agents or compositions.

In another embodiment, the present invention also provides the use of an active ingredient selected from among:
(a) a peptide of the present invention,
(b) a nucleic acid encoding such a peptide as disclosed herein in an expressible form,
(c) an APC of the present invention, and
(d) a cytotoxic T cells of the present invention in manufacturing a pharmaceutical composition or agent for treating cancer.

Alternatively, the present invention further provides an active ingredient selected from among:
(a) a peptide of the present invention,
(b) a nucleic acid encoding such a peptide as disclosed herein in an expressible form,
(c) an APC of the present invention, and
(d) a cytotoxic T cells of the present invention for use in treating cancer.

Alternatively, the present invention further provides a method or process for manufacturing a pharmaceutical composition or agent for treating cancer, wherein the method or process includes the step of formulating a pharmaceutically or physiologically acceptable carrier with an active ingredient selected from among:
(a) a peptide of the present invention,
(b) a nucleic acid encoding such a peptide as disclosed herein in an expressible form,
(c) an APC of the present invention, and
(d) a cytotoxic T cells of the present invention as active ingredients.

In another embodiment, the present invention also provides a method or process for manufacturing a pharmaceutical composition or agent for treating cancer, wherein the method or process includes the step of admixing an active ingredient with a pharmaceutically or physiologically acceptable carrier, wherein the active ingredient is selected from among:
(a) a peptide of the present invention,
(b) a nucleic acid encoding such a peptide as disclosed herein in an expressible form,
(c) an APC of the present invention, and
(d) a cytotoxic T cells of the present invention.

Alternatively, the pharmaceutical composition or agent of the present invention may be used for either or both the prophylaxis of cancer and prevention of postoperative recurrence thereof.

The present pharmaceutical agents or compositions find use as a vaccine. In the context of the present invention, the phrase “vaccine” (also referred to as an “immunogenic composition”) refers to a substance that has the function to induce anti-tumor immunity upon inoculation into animals.

The pharmaceutical agents or compositions of the present invention can be used to treat and/or prevent cancers, and/or prevention of postoperative recurrence thereof in subjects or patients including human and any other mammal including, but not limited to, mouse, rat, guinea-pig, rabbit, cat, dog, sheep, goat, pig, cattle, horse, monkey, baboon, and chimpanzee, particularly a commercially important animal or a domesticated animal.

According to the present invention, polypeptides having an amino acid sequence selected from among SEQ ID NOs: 2, 4, 7, 21, 25, 29, 32, 35, 37, 40, 49, 53, 55, 56, 57, 62, 63 and 67 or polypeptides having an amino acid sequence selected from among SEQ ID NOs: 75, 85, 99, 101, 111, 114, 121, 125, 130, 139, 140, 141, 142, 143, 145, 148 and 150 have been found to be HLA-A24 or HLA-A02 restricted epitope peptides or candidates that can induce potent and specific immune response. Therefore, the present pharmaceutical agents or compositions which include any of these polypeptides with the amino acid sequences of SEQ ID NOs: 2, 4, 7, 21, 25, 29, 32, 35, 37, 40, 49, 53, 55, 56, 57, 62, 63 and 67 are particularly suited for the administration to subjects whose HLA antigen is HLA-A24. On the one hand, the present pharmaceutical agents or compositions which contain any of these polypeptides having the amino acid sequences of SEQ ID NOs: 75, 85, 99, 101, 111, 114, 121, 125, 130, 139, 140, 141, 142, 143, 145, 148 and 150 are particularly suited for the administration to subjects whose HLA antigen is HLA-A02. The same applies to pharmaceutical agents or compositions which contain polynucleotides encoding any of these polypeptides.

Cancers to be treated by the pharmaceutical agents or compositions of the present invention are not limited and include all kinds of cancers wherein IQGAP3 is involved, including for example, renal, esophageal, gastric, lung, breast, bladder and pancreatic cancer.

The present pharmaceutical agents or compositions can contain in addition to the aforementioned active ingredients, other peptides which have the ability to induce CTLs against cancerous cells, other polynucleotides encoding the other peptides, other cells that present the other peptides, or such. Herein, the other peptides that have the ability to induce CTLs against cancerous cells are exemplified by cancer specific antigens (e.g., identified TAAs), but are not limited thereto.

If needed, the pharmaceutical agents or compositions of the present invention can optionally include other therapeutic substances as an active ingredient, so long as the substance does not inhibit the antitumoral effect of the active ingredient, e.g., any of the present peptides. For example, formulations can include anti-inflammatory agents or compositions, pain killers, chemotherapeutics, and the like. In addition to including other therapeutic substances in the medicament itself, the medicaments of the present invention can also be administered sequentially or concurrently with the one or more other pharmacologic agents or compositions. The amounts of medicament and pharmacologic agent or compositions depend, for example, on what type of pharmacologic agent(s) or composition(s) is/are used, the disease being treated, and the scheduling and routes of administration.

It should be understood that, in addition to the ingredients particularly mentioned herein, the pharmaceutical agents or compositions of the present invention can include other agents or compositions conventional in the art having regard to the type of formulation in question.

In one embodiment of the present invention, the present pharmaceutical agents or compositions can be included in articles of manufacture and kits containing materials useful for treating the pathological conditions of the disease to be treated, e.g., cancer. The article of manufacture can include a container of any of the present pharmaceutical agents or compositions with a label. Suitable containers include bottles, vials, and test tubes. The containers can be formed from a variety of materials, such as glass or plastic. The label on the container should indicate the agent or composition is used for treating or prevention of one or more conditions of the disease. The label can also indicate directions for administration and so on.

In addition to the container described above, a kit including a pharmaceutical agent or composition of the present invention can optionally further include a second container housing a pharmaceutically-acceptable diluent. It can further include other materials desirable from a commercial and user standpoint, including other buffers, diluents, filters, needles, syringes, and package inserts with instructions for use.

The pharmaceutical compositions can, if desired, be presented in a pack or dispenser device which can contain one or more unit dosage forms containing the active ingredient. The pack can, for example, include metal or plastic foil, such as a blister pack. The pack or dispenser device can be accompanied by instructions for administration.

(1) Pharmaceutical Agents or Compositions Containing the Peptides as the Active Ingredient

The peptides of the present invention can be administered directly as a pharmaceutical agent or composition, or if necessary, that has been formulated by conventional formulation methods. In the latter case, in addition to the peptides of the present invention, carriers, excipients, and such that are ordinarily used for drugs can be included as appropriate without particular limitations. Examples of such carriers are sterilized water, physiological saline, phosphate buffer, culture fluid and such. Furthermore, the pharmaceutical agents or compositions can contain as necessary, stabilizers, suspensions, preservatives, surfactants and such. The pharmaceutical agents or compositions of the present invention can be used for anticancer purposes.

The peptides of the present invention can be prepared as a combination, composed of two or more of peptides of the invention, to induce CTL in vivo. The peptide combination can take the form of a cocktail or can be conjugated to each other using standard techniques. For example, the peptides can be chemically linked or expressed as a single fusion polypeptide sequence. The peptides in the combination can be the same or different. By administering the peptides of the present invention, the peptides are presented at a high density by the HLA antigens on APCs, then CTLs that specifically react toward the complex formed between the displayed peptide and the HLA antigen are induced. Alternatively, APCs that present any of the peptides of the present invention on their cell surface are obtained by removing APCs (e.g., DCs) from the subjects, which are stimulated by the peptides of the present invention, CTL is induced in the subjects by readministering these APCs (e.g., DCs) to the subjects, and as a result, aggressiveness towards the cancer cells can be increased.

The pharmaceutical agents or compositions for the treatment and/or prevention of cancer, which include a peptide of the present invention as the active ingredient, can also include an adjuvant known to effectively establish cellular immunity. Alternatively, the pharmaceutical agents or compositions can be administered with other active ingredients or administered by formulation into granules. An adjuvant refers to a compound that enhances the immune response against the protein when administered together (or successively) with the protein having immunological activity. Adjuvants contemplated herein include those described in the literature (Clin Microbiol Rev 1994, 7: 277-89). Examples of suitable adjuvants include, but are not limited to, aluminum phosphate, aluminum hydroxide, alum, cholera toxin, salmonella toxin, and such, but are not limited thereto.

Furthermore, liposome formulations, granular formulations in which the peptide is bound to few-micrometers diameter beads, and formulations in which a lipid is bound to the peptide may be conveniently used.

In some embodiments, the pharmaceutical agents or compositions of the invention may further include a component which primes CTL. Lipids have been identified as agents or compositions capable of priming CTL in vivo against viral antigens. For example, palmitic acid residues can be attached to the epsilon- and alpha-amino groups of a lysine residue and then linked to a peptide of the invention. The lipidated peptide can then be administered either directly in a micelle or particle, incorporated into a liposome, or emulsified in an adjuvant. As another example of lipid priming of CTL responses, E. coli lipoproteins, such as tripalmitoyl-S-glycerylcysteinlyseryl-serine (P3CSS) can be used to prime CTL when covalently attached to an appropriate peptide (see, e.g., Deres et al., Nature 1989, 342: 561-4).

The method of administration can be oral, intradermal, subcutaneous, intravenous injection, or such, and systemic administration or local administration to the vicinity of the targeted sites. The administration can be performed by single administration or boosted by multiple administrations. The dose of the peptides of the present invention can be adjusted appropriately according to the disease to be treated, age of the patient, weight, method of administration, and such, and is ordinarily 0.001 mg to 1000 mg, for example, 0.001 mg to 1000 mg, for example, 0.1 mg to 10 mg, and can be administered once in a few days to few months. One skilled in the art can appropriately select a suitable dose.

(2) Pharmaceutical Agents or Compositions Containing Polynucleotides as the Active Ingredient

The pharmaceutical agents or compositions of the invention can also contain nucleic acids encoding the peptides disclosed herein in an expressible form. Herein, the phrase “in an expressible form” means that the polynucleotide, when introduced into a cell, will be expressed in vivo as a polypeptide that induces anti-tumor immunity. In an exemplified embodiment, the nucleic acid sequence of the polynucleotide of interest includes regulatory elements necessary for expression of the polynucleotide. The polynucleotide(s) can be equipped so to achieve stable insertion into the genome of the target cell (see, e.g., Thomas K R & Capecchi M R, Cell 1987, 51: 503-12 for a description of homologous recombination cassette vectors). See, e.g., Wolff et al., Science 1990, 247: 1465-8; U.S. Pat. Nos. 5,580,859; 5,589,466; 5,804,566; 5,739,118; 5,736,524; 5,679,647; and WO 98/04720. Examples of DNA-based delivery technologies include “naked DNA”, facilitated (bupivacaine, polymers, peptide-mediated) delivery, cationic lipid complexes, and particle-mediated (“gene gun”) or pressure-mediated delivery (see, e.g., U.S. Pat. No. 5,922,687). The peptides of the present invention can also be expressed by viral or bacterial vectors. Examples of expression vectors include attenuated viral hosts, such as vaccinia or fowlpox. This approach involves the use of vaccinia virus, e.g., as a vector to express nucleotide sequences that encode the peptide. Upon introduction into a host, the recombinant vaccinia virus expresses the immunogenic peptide, and thereby elicits an immune response. Vaccinia vectors and methods useful in immunization protocols are described in, e.g., U.S. Pat. No. 4,722,848. Another vector is BCG (Bacille Calmette Guerin). BCG vectors are described in Stover et al., Nature 1991, 351: 456-60. A wide variety of other vectors useful for therapeutic administration or immunization e.g., adeno and adeno-associated virus vectors, retroviral vectors, Salmonella typhi vectors, detoxified anthrax toxin vectors, and the like, will be apparent. See, e.g., Shata et al., Mol Med Today 2000, 6: 66-71; Shedlock et al., J Leukoc Biol 2000, 68: 793-806; Hipp et al., In Vivo 2000, 14: 571-85.

Delivery of a polynucleotide into a subject can be either direct, in which case the subject is directly exposed to a polynucleotide-carrying vector, or indirect, in which case, cells are first transformed with the polynucleotide of interest in vitro, then the cells are transplanted into the subject. Theses two approaches are known, respectively, as in vivo and ex vivo gene therapies.

For general reviews of the methods of gene therapy, see Goldspiel et al., Clinical Pharmacy 1993, 12: 488-505; Wu and Wu, Biotherapy 1991, 3: 87-95; Tolstoshev, Ann Rev Pharmacol Toxicol 1993, 33: 573-96; Mulligan, Science 1993, 260: 926-32; Morgan & Anderson, Ann Rev Biochem 1993, 62: 191-217; Trends in Biotechnology 1993, 11(5): 155-215). Methods commonly known in the art of recombinant DNA technology which can also be used for the present invention are described in eds.

Ausubel et al., Current Protocols in Molecular Biology, John Wiley & Sons, NY, 1993; and Krieger, Gene Transfer and Expression, A Laboratory Manual, Stockton Press, NY, 1990.

The method of administration can be oral, intradermal, subcutaneous, intravenous injection, or such, and systemic administration or local administration to the vicinity of the targeted sites finds use. The administration can be performed by single administration or boosted by multiple administrations. The dose of the polynucleotide in the suitable carrier or cells transformed with the polynucleotide encoding the peptides of the present invention can be adjusted appropriately according to the disease to be treated, age of the patient, weight, method of administration, and such, and is ordinarily 0.001 mg to 1000 mg, for example, 0.001 mg to 1000 mg, for example, 0.1 mg to 10 mg, and can be administered once every a few days to once every few months. One skilled in the art can appropriately select the suitable dose.

### IX. METHODS USING THE PEPTIDES, EXOSOMES, APCs AND CTLs

The peptides of the present invention and polynucleotides encoding such peptides can be used for inducing APCs and CTLs. The exosomes and APCs of the present invention can be also used for inducing CTLs. The peptides, polynucleotides, exosomes and APCs can be used in combination with any other compounds so long as the compounds do not inhibit their CTL inducibility. Thus, any of the aforementioned pharmaceutical agents or compositions of the present invention can be used for inducing CTLs, and in addition thereto, those including the peptides and polynucleotides can be also be used for inducing APCs as discussed below.

(1) Method of Inducing Antigen-Presenting Cells (APCs)

The present invention provides methods of inducing APCs using the peptides of the present invention or polynucleotides encoding the peptides. The induction of APCs can be performed as described above in section “VI. Antigen-presenting cells”. The present invention also provides a method for inducing APCs having a high level of CTL inducibility, the induction of which has been also mentioned under the item of “VI. Antigen-presenting cells”, supra.

(2) Method of Inducing CTLs

Furthermore, the present invention provides methods for inducing CTLs using the peptides of the present invention, polynucleotides encoding the peptides, exosomes or APCs presenting the peptides.

The present invention also provides methods for inducing CTLs using a polynucleotide encoding a polypeptide that is capable of forming a T cell receptor (TCR) subunit recognizing a complex of the peptides of the present invention and HLA antigen. Preferably, the methods for inducing CTLs comprise at least one step selected from the group consisting of:

a: contacting a CD8-positive T cell with an antigen-presenting cell and/or an exosome that presents on its surface a complex of an HLA antigen and the peptide of the present invention, and
b: introducing a polynucleotide encoding a polypeptide that is capable of forming a TCR subunit recognizing a complex of the peptide of the present invention and HLA antigen into a CD8 positive T cell.

When the peptides of this invention are administered to a subject, CTL is induced in the body of the subject, and the strength of the immune response targeting the tumor-associated endothelia is enhanced. Alternatively, the peptides and polynucleotides encoding the peptides can be used for an ex vivo therapeutic method, in which subject-derived APCs, and CD8-positive cells, or peripheral blood mononuclear leukocytes are contacted (stimulated) with the peptides of the present invention in vitro, and after inducing CTL, the activated CTL cells are returned to the subject. For example, the method can include the steps of:

a: collecting APCs from subject:,
b: contacting with the APCs of step a, with the peptide:,
c: mixing the APCs of step b with CD⁸⁺ T cells, and co-culturing for inducing CTLs: and
d: collecting CD⁸⁺ T cells from the co-culture of step c.

Alternatively, according to the present invention, use of the peptides of the present invention for manufacturing a pharmaceutical composition inducing CTLs is provided. Further, the present invention also provides the peptide of the present invention for inducing CTLs.

The CD⁸⁺ T cells having cytotoxic activity obtained by step d can be administered to the subject as a vaccine. The APCs to be mixed with the CD⁸⁺ T cells in above step c can also be prepared by transferring genes coding for the present peptides into the APCs as detailed above in section “VI. Antigen-presenting cells”; but are not limited thereto. Accordingly, any APC or exosome which effectively presents the present peptides to the T cells can be used for the present method.

The following examples are presented to illustrate the present invention and to assist one of ordinary skill in making and using the same. The examples are not intended in any way to otherwise limit the scope of the invention.

### EXAMPLES

Materials and Methods

Cell Lines

A24 lymphoblastoid cell line (A24LCL) cells were established by transformation with Epstein-bar virus into HLA-A24 positive human B lymphocyte. T2 (HLA-A2), human B-lymphoblastoid cell line, and COST were purchased from ATCC.

Candidate Selection of Peptides Derived from IQGAP3

9-mer and 10-mer peptides derived from IQGAP3 that bind to HLA-A*2402 and HLA-A*0201 molecules were predicted using binding prediction software “BIMAS” (http://www-bimas.cit.nih.gov/molbio/hla_bind), which algorithms had been described by Parker K C et al. (J Immunol 1994, 152(1): 163-75) and Kuzushima K et al. (Blood 2001, 98(6): 1872-81). These peptides were synthesized by Sigma (Sapporo, Japan) according to a standard solid phase synthesis method and purified by reversed phase high performance liquid chromatography (HPLC). The purity (>90%) and the identity of the peptides were determined by analytical HPLC and mass spectrometry analysis, respectively. Peptides were dissolved in dimethylsulfoxide (DMSO) at 20 mg/ml and stored at −80 degrees C.

In Vitro CTL Induction

Monocyte-derived dendritic cells (DCs) were used as antigen-presenting cells (APCs) to induce cytotoxic T lymphocyte (CTL) responses against peptides presented on human leukocyte antigen (HLA). DCs were generated in vitro as described elsewhere (Nakahara S et al., Cancer Res 2003 Jul. 15, 63(14): 4112-8). Specifically, peripheral blood mononuclear cells (PBMCs) isolated from a normal volunteer (HLA-A*2402 or HLA-A*0201 positive) by Ficoll-Plaque (Pharmacia) solution were separated by adherence to a plastic tissue culture dish (Becton Dickinson) so as to enrich them as the monocyte fraction. The monocyte-enriched population was cultured in the presence of 1000 U/ml of granulocyte-macrophage colony-stimulating factor (GM-CSF) (R&D System) and 1000 U/ml of interleukin (IL)-4 (R&D System) in AIM-V Medium (Invitrogen) containing 2% heat-inactivated autologous serum (AS). After 7 days of culture, the cytokine-induced DCs were pulsed with 20 mcg/ml of each of the synthesized peptides in the presence of 3 mcg/ml of beta2-microglobulin for 3 hr at 37 degrees C. in AIM-V Medium. The generated cells appeared to express DC-associated molecules, such as CD80, CD83, CD86 and HLA class II, on their cell surfaces (data not shown). These peptide-pulsed DCs were then inactivated by Mitomycin C (MMC) (30 mcg/ml for 30 min) and mixed at a 1:20 ratio with autologous CD8+ T cells, obtained by positive selection with CD8 Positive Isolation Kit (Dynal). These cultures were set up in 48-well plates (Corning); each well contained 1.5×10⁴ peptide-pulsed DCs, 3×10⁵ CD8+ T cells and 10 ng/ml of IL-7 (R&D System) in 0.5 ml of AIM-V/2% AS medium. Three days later, these cultures were supplemented with IL-2 (CHIRON) to a final concentration of 20 IU/ml. On day 7 and 14, the T cells were further stimulated with the autologous peptide-pulsed DCs. The DCs were prepared each time by the same way described above. CTL was tested against peptide-pulsed A24LCL cells after the 3rd round of peptide stimulation on day 21 (Tanaka H et al., Br J Cancer 2001 Jan. 5, 84(1): 94-9; Umano Y et al., Br J Cancer 2001 Apr. 20, 84(8): 1052-7; Uchida N et al., Clin Cancer Res 2004 Dec. 15, 10(24): 8577-86; Suda T et al., Cancer Sci 2006 May, 97(5): 411-9; Watanabe T et al., Cancer Sci 2005 August, 96(8): 498-506).

CTL Expansion Procedure

CTLs were expanded in culture using the method similar to the one described by Riddell et al. (Walter E A et al., N Engl J Med 1995 Oct. 19, 333(16): 1038-44; Riddell S R et al., Nat Med 1996 February, 2(2): 216-23). A total of 5×10⁴ CTLs were suspended in 25 ml of AIM-V/5% AS medium with 2 kinds of human B-lymphoblastoid cell lines, inactivated by MMC, in the presence of 40 ng/ml of anti-CD3 monoclonal antibody (Pharmingen). One day after initiating the cultures, 120 IU/ml of IL-2 were added to the cultures. The cultures were fed with fresh AIM-V/5% AS medium containing 30 IU/ml of IL-2 on days 5, 8 and 11 (Tanaka H et al., Br J Cancer 2001 Jan. 5, 84(1): 94-9; Umano Y et al., Br J Cancer 2001 Apr. 20, 84(8): 1052-7; Uchida N et al., Clin Cancer Res 2004 Dec. 15, 10(24): 8577-86; Suda T et al., Cancer Sci 2006 May, 97(5): 411-9; Watanabe T et al., Cancer Sci 2005 August, 96(8): 498-506).

Establishment of CTL Clones

The dilutions were made to have 0.3, 1, and 3 CTLs/well in 96 round-bottomed micro titer plate (Nalge Nunc International). CTLs were cultured with 1×10⁴ cells/well of 2 kinds of human B-lymphoblastoid cell lines, 30 ng/ml of anti-CD3 antibody, and 125 U/ml of IL-2 in a total of 150 and/well of AIM-VMedium containing 5% AS. 50 mcl/well of IL-2 were added to the medium 10 days later so to reach a final concentration of 125 U/ml IL-2. CTL activity was tested on the 14th day, and CTL clones were expanded using the same method as described above (Uchida N et al., Clin Cancer Res 2004 Dec. 15, 10(24): 8577-86; Suda T et al., Cancer Sci 2006 May, 97(5): 411-9; Watanabe T et al., Cancer Sci 2005 August, 96(8): 498-506).

Specific CTL Activity

To examine specific CTL activity, interferon (IFN)-gamma enzyme-linked immunospot (ELISPOT) assay and IFN-gamma enzyme-linked immunosorbent assay (ELISA) were performed. Specifically, peptide-pulsed A24 or T2 LCL (1×10⁴/well) was prepared as stimulator cells. Cultured cells in 48 wells were used as responder cells. IFN-gamma ELISPOT assay and IFN-gamma ELISA assay were performed under manufacture procedure.

Establishment of the Cells Forcibly Expressing Either or Both of the Target Gene and HLA-A24

The cDNA encoding an open reading frame of target gene or HLA-A24 was amplified by PCR. The PCR-amplified product was cloned into pCAGGS vector. The plasmids were transfected into COS7, which is the target gene and HLA-A24-null cell line, using lipofectamine 2000 (Invitrogen) according to the manufacturer's recommended procedures. After 2 days from transfection, the transfected cells were harvested with versene (Invitrogen) and used as the target cells (5×10⁴ cells/well) for CTL activity assay.

Plasmid Transfection

The cDNA encoding an open reading frame of target genes or HLA-A*0201 was amplified by PCR. The PCR-amplified products were cloned into pCAGGS vector. The plasmids were transfected into COST, which is the target genes and HLA-A*0201-negative cell line, using lipofectamine 2000 (Invitrogen) according to the manufacturer's recommended procedures. After 2 days from transfection, the transfected cells were harvested with versene (Invitrogen) and used as the target cells (5×10⁴ cells/well) for CTL activity assay

Results

Prediction of HLA-A24 Binding Peptides Derived from IQGAP3

Table 1 shows the HLA-A*2402 binding peptides of IQGAP3 in order of highest binding affinity. Table 1a shows the 9mer peptides and Table 1b shows the 10mer peptides derived from IQGAP3. Total of 68 peptides having potential HLA-A24 binding ability were selected and examined to determine the epitope peptides.

TABLE 1a
Table 1a; HLA-A24 binding 9mer
peptides derived from IQGAP3

| Start | Amino acid | Binding | SEQ ID |
|---|---|---|---|
| Position | sequence | Score | NO. |
| 483 | RYFDALLKL | 528 | 1 |
| 955 | AYQHLFYLL | 432 | 2 |
| 1458 | GYQGLVDEL | 396 | 3 |
| 1167 | VYKVVGNLL | 336 | 4 |
| 92 | RYQATGLHF | 300 | 5 |
| 417 | MYQLELAVL | 300 | 6 |
| 779 | IYLEWLQYF | 216 | 7 |
| 139 | VYCIHALSL | 200 | 8 |
| 181 | KYGLQLPAF | 200 | 9 |
| 773 | GYRQRKIYL | 200 | 10 |
| 809 | QYLRRLHYF | 150 | 11 |
| 680 | AYYFHLQTF | 120 | 12 |
| 960 | FYLLQTQPI | 90 | 13 |
| 1588 | RFQLHYQDL | 72 | 14 |
| 1574 | KFEVNAKFL | 60 | 15 |
| 749 | KFAEHSHFL | 48 | 16 |
| 1621 | IFLLNKKFL | 30 | 17 |
| 867 | DFLAEAELL | 30 | 18 |
| 188 | AFSKIGGIL | 28 | 19 |
| 1224 | AFSGQSQHL | 24 | 20 |
| 74 | CFAPSVVPL | 24 | 21 |
| 1145 | RYVAKVLKA | 16.5 | 22 |
| 835 | KAQDDYRIL | 14.4 | 23 |
| 1486 | KLQATLQGL | 14.4 | 24 |
| 26 | RQNVAYQYL | 14.4 | 25 |
| 1439 | RVLRNLRRL | 12 | 26 |
| 1423 | RSLTAHSLL | 12 | 27 |
| 564 | RYHLLLVAA | 12 | 28 |
| 137 | RVVYCIHAL | 12 | 29 |
| 1442 | RNLRRLEAL | 12 | 30 |
| 1436 | KQRRVLRNL | 11.2 | 31 |
| 63 | RNGVLLAKL | 10.56 | 32 |
| 1279 | VYITVGELV | 10.5 | 33 |
| 896 | NIMDIKIGL | 10.08 | 34 |

TABLE 1b
Table 1b; HLA-A24 binding 10mer
peptides derived from IQGAP3

| Start | Amino acid | Binding | SEQ ID |
|---|---|---|---|
| Position | sequence | Score | NO. |
| 1600 | QYEGVAVMKL | 330 | 35 |
| 1510 | QYIRACLDHL | 300 | 36 |
| 1507 | YYSQYIRACL | 280 | 37 |
| 1237 | DYLEETHLKF | 198 | 38 |
| 984 | KFMEAVIFSL | 100.8 | 39 |
| 139 | VYCIHALSLF | 100 | 40 |
| 1588 | RFQLHYQDLL | 60 | 41 |
| 815 | HYFQKNVNSI | 60 | 42 |
| 785 | QYFKANLDAI | 50 | 43 |
| 968 | IYLAKLIFQM | 45 | 44 |
| 649 | GYQRALESAM | 45 | 45 |
| 12 | AYERLTAEEM | 41.25 | 46 |
| 732 | GFVIQLQARL | 36 | 47 |
| 1580 | KFLGVDMERF | 30 | 48 |
| 1097 | PYDVTPEQAL | 24 | 49 |
| 329 | DFADWYLEQL | 24 | 50 |
| 1145 | RYVAKVLKAT | 21 | 51 |
| 886 | RSNQQLEQDL | 17.28 | 52 |
| 345 | KAQELGLVEL | 15.84 | 53 |
| 1047 | RGQSALQEIL | 14.4 | 54 |
| 1614 | KVNVNLLIFL | 14.4 | 55 |
| 191 | KIGGILANEL | 12.672 | 56 |
| 314 | KALQDPALAL | 12 | 57 |
| 1545 | KGVLVEIEDL | 12 | 58 |
| 630 | RVLRNPAVAL | 12 | 59 |
| 181 | KYGLQLPAFS | 12 | 60 |
| 728 | KANVGFVIQL | 12 | 61 |
| 1363 | RSLLLSTKQL | 12 | 62 |
| 1114 | RLDIALRNLL | 11.52 | 63 |
| 1592 | HYQDLLQLQY | 10.8 | 64 |
| 1458 | GYQGLVDELA | 10.5 | 65 |
| 295 | GALEVVDDAL | 10.08 | 66 |
| 1207 | HALGAVAQLL | 10.08 | 67 |
| 99 | HFRHTDNINF | 10 | 68 |

Start position indicates the number of amino acid residue from the N-terminal of IQGAP3.
Binding score is derived from “BIMAS”.

CTL Induction with the Predicted Peptides from IQGAP3 Restricted with HLA-A*2402 and Establishment for CTL Lines Stimulated with IQGAP3 Derived Peptides

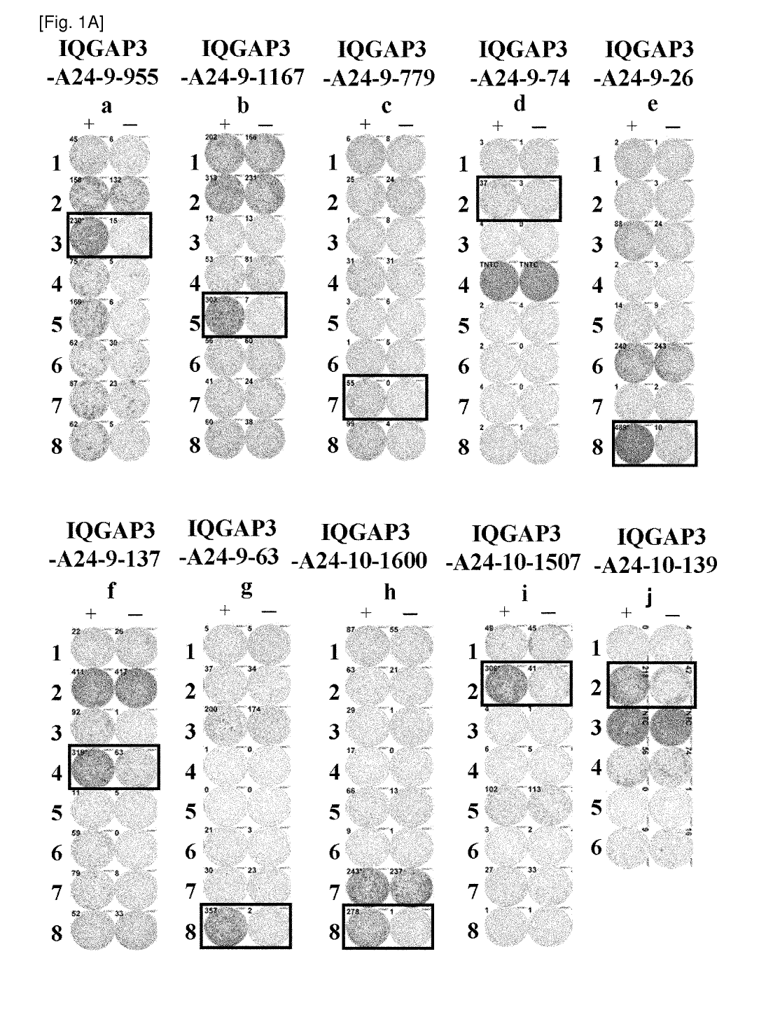

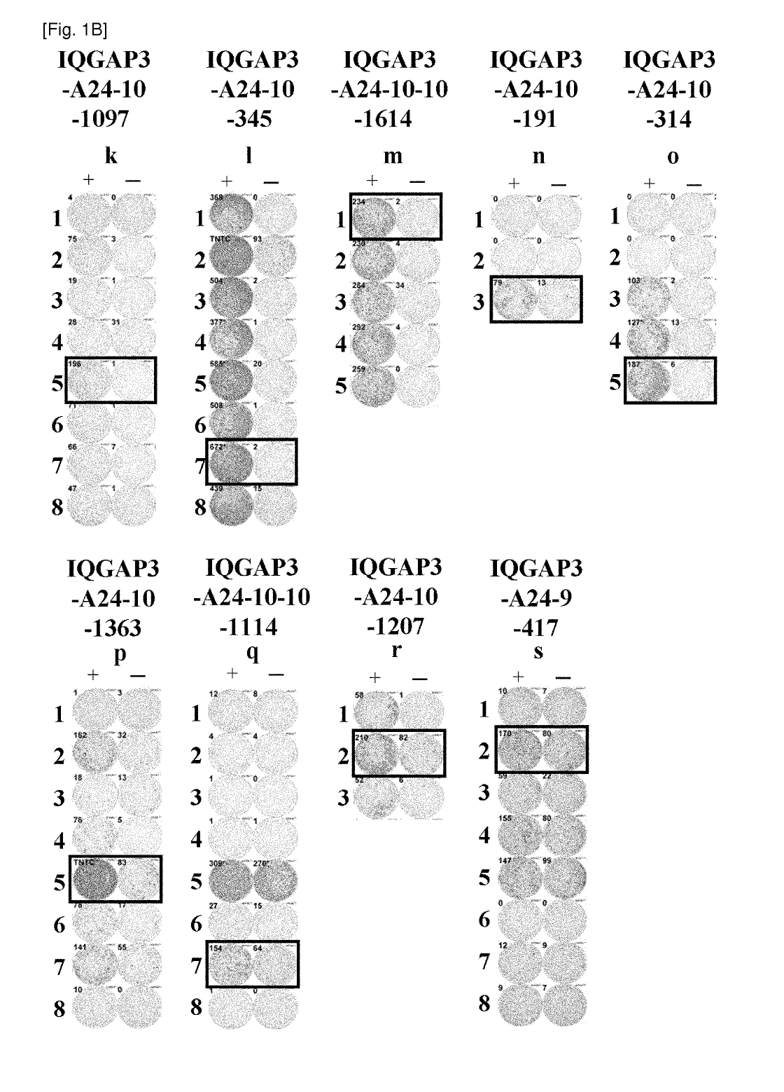

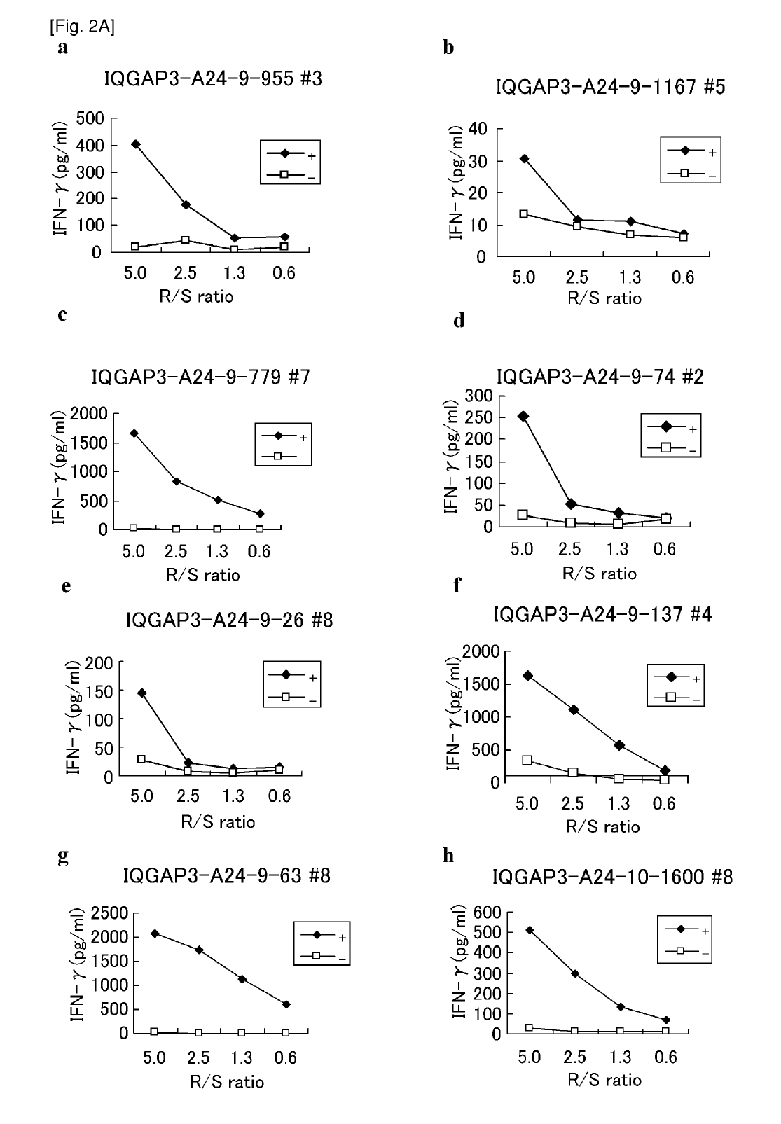

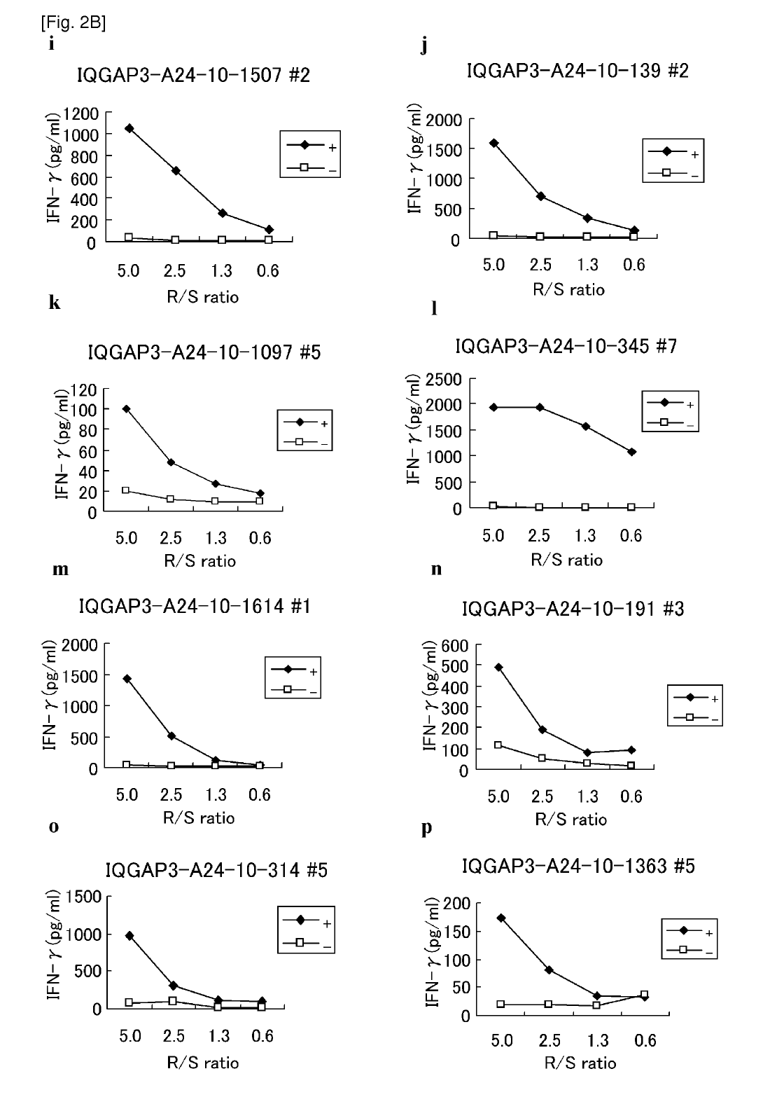

CTLs for those peptides derived from IQGAP3 were generated according to the protocols as described in “Materials and Methods”. Peptide specific CTL activity was determined by IFN-gamma ELISPOT assay (FIG. 1a-r). It showed that IQGAP3-A24-9-955 (SEQ ID NO:2) (a), IQGAP3-A24-9-1167 (SEQ ID NO:4) (b), IQGAP3-A24-9-779 (SEQ ID NO:7) (c), IQGAP3-A24-9-74 (SEQ ID NO: 21) (d), IQGAP3-A24-9-26 (SEQ ID NO:25) (e), IQGAP3-A24-9-137 (SEQ ID NO:29) (f), IQGAP3-A24-9-63 (SEQ ID NO:32) (g), IQGAP3-A24-10-1600 (SEQ ID NO:35) (h), IQGAP3-A24-10-1507 (SEQ ID NO:37) (i), IQGAP3-A24-10-139 (SEQ ID NO: 40) (j), IQGAP3-A24-10-1097 (SEQ ID NO:49) (k), IQGAP3-A24-10-345 (SEQ ID NO:53) (l), IQGAP3-A24-10-1614 (SEQ ID NO:55) (m), IQGAP3-A24-10-191 (SEQ ID NO:56) (n), IQGAP3-A24-10-314 (SEQ ID NO:57) (o), IQGAP3-A24-10-1363 (SEQ ID NO:62) (p), IQGAP3-A24-10-1114 (SEQ ID NO:63) (q) and IQGAP3-A24-10-1207 (SEQ ID NO: 67) (r) demonstrated potent IFN-gamma production as compared to the control wells. Furthermore, the cells in the positive well number #3 and 6 stimulated with IQGAP3-A24-9-955 (SEQ ID NO:2) (a), #5 with IQGAP3-A24-9-1167 (SEQ ID NO:4) (b), #7 with IQGAP3-A24-9-779 (SEQ ID NO:7) (c), #2 with IQGAP3-A24-9-74 (SEQ ID NO: 21) (d), #8 with IQGAP3-A24-9-26 (SEQ ID NO:25) (e), #4 with IQGAP3-A24-9-137 (SEQ ID NO:29) (f), #8 with IQGAP3-A24-9-63 (SEQ ID NO:32) (g), #8 with IQGAP3-A24-10-1600 (SEQ ID NO:35) (h), #2 with IQGAP3-A24-10-1507 (SEQ ID NO:37) (i), #2 with IQGAP3-A24-10-139 (SEQ ID NO: 40) (j), #5 with IQGAP3-A24-10-1097 (SEQ ID NO:49) (k), #7 with IQGAP3-A24-10-345 (SEQ ID NO:53) (l), #1 with IQGAP3-A24-10-1614 (SEQ ID NO:55) (m), #3 with IQGAP3-A24-10-191 (SEQ ID NO:56) (n), #5 with IQGAP3-A24-10-314 (SEQ ID NO:57) (o), #5 with IQGAP3-A24-10-1363 (SEQ ID NO:62) (p), #7 with IQGAP3-A24-10-1114 (SEQ ID NO:63) (q) and #2 with IQGAP3-A24-10-1207 (SEQ ID NO: 67) (r) were expanded and established CTL lines. CTL activity of those CTL lines was determined by IFN-gamma ELISA assay (FIG. 2a-r). It showed that all CTL lines demonstrated potent IFN-gamma production against the target cells pulsed with corresponding peptide as compared to target cells without peptide pulse. On the other hand, no CTL lines could be established by stimulation with other peptides shown in Table 1, despite those peptide had possible binding activity with HLA-A*2402. For example, typical negative data of CTL response stimulated with IQGAP3-A24-9-417 (SEQ ID NO: 6) was shown in FIG. 1(s) and FIG. 2(s). The results herein indicate that eighteen peptides derived from IQGAP3 and screened as the peptides could induce potent CTL lines.

Specific CTL Activity Against Target Cells Exogenously Expressing IQGAP3 and HLA-A*2402

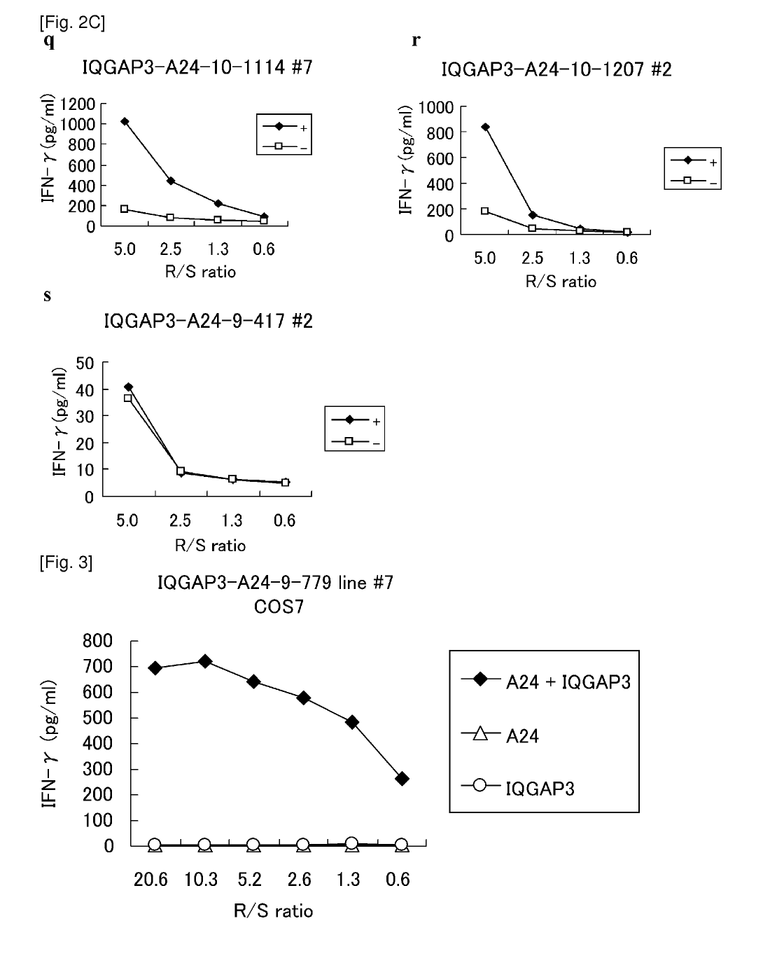

The established CTL lines raised against these peptides were examined for their ability to recognize target cells that endogenously express IQGAP3 and HLA-A*2402 molecule. Specific CTL activity against COS7 cells which transfected with both the full length of IQGAP3 and HLA-A*2402 molecule gene (a specific model for the target cells that endogenously express IQGAP3 and HLA-A*2402 gene) was tested using the CTL lines raised by corresponding peptide as the effecter cells. COS7 cells transfected with either full length of IQGAP3 gene or HLA-A*2402 were prepared as control. In FIG. 3, the CTLs stimulated with IQGAP3-A24-9-779 (SEQ ID NO: 7) showed potent CTL activity against COS7 cells expressing both IQGAP3 and HLA-A*2402. On the other hand, no significant specific CTL activity was detected against the controls. Thus, these data clearly demonstrate that IQGAP3-A24-9-779 (SEQ ID NO: 7) is naturally expressed on the target cells with HLA-A*2402 molecule and recognized by the CTLs. These results indicate that this peptide derived from IQGAP3 may be available to apply the cancer vaccines for patients with IQGAP3 expressing tumors.

Homology Analysis of Antigen Peptides

The CTLs stimulated with IQGAP3-A24-9-955 (SEQ ID NO:2), IQGAP3-A24-9-1167 (SEQ ID NO:4), IQGAP3-A24-9-779 (SEQ ID NO:7), IQGAP3-A24-9-74 (SEQ ID NO: 21), IQGAP3-A24-9-26 (SEQ ID NO:25), IQGAP3-A24-9-137 (SEQ ID NO:29), IQGAP3-A24-9-63 (SEQ ID NO:32), IQGAP3-A24-10-1600 (SEQ ID NO:35), IQGAP3-A24-10-1507 (SEQ ID NO:37), IQGAP3-A24-10-139 (SEQ ID NO: 40), IQGAP3-A24-10-1097 (SEQ ID NO:49), IQGAP3-A24-10-345 (SEQ ID NO:53), IQGAP3-A24-10-1614 (SEQ ID NO:55), IQGAP3-A24-10-191 (SEQ ID NO:56), IQGAP3-A24-10-314 (SEQ ID NO:57), IQGAP3-A24-10-1363 (SEQ ID NO:62), IQGAP3-A24-10-1114 (SEQ ID NO:63) and IQGAP3-A24-10-1207 (SEQ ID NO: 67) showed significant and specific CTL activity. This result may be due to the fact that the sequences of IQGAP3-A24-9-955 (SEQ ID NO:2), IQGAP3-A24-9-1167 (SEQ ID NO:4), IQGAP3-A24-9-779 (SEQ ID NO:7), IQGAP3-A24-9-74 (SEQ ID NO: 21), IQGAP3-A24-9-26 (SEQ ID NO:25), IQGAP3-A24-9-137 (SEQ ID NO:29), IQGAP3-A24-9-63 (SEQ ID NO:32), IQGAP3-A24-10-1600 (SEQ ID NO:35), IQGAP3-A24-10-1507 (SEQ ID NO:37), IQGAP3-A24-10-139 (SEQ ID NO: 40), IQGAP3-A24-10-1097 (SEQ ID NO:49), IQGAP3-A24-10-345 (SEQ ID NO:53), IQGAP3-A24-10-1614 (SEQ ID NO:55), IQGAP3-A24-10-191 (SEQ ID NO:56), IQGAP3-A24-10-314 (SEQ ID NO:57), IQGAP3-A24-10-1363 (SEQ ID NO:62), IQGAP3-A24-10-1114 (SEQ ID NO:63) and IQGAP3-A24-10-1207 (SEQ ID NO: 67) are homologous to peptides derived from other molecules that are known to sensitize the human immune system. To exclude this possibility, homology analyses were performed for these peptide sequences using as queries the BLAST algorithm (http://www.ncbi.nlm.nih.gov/blast/blast.cgi) which revealed no sequence with significant homology. The results of homology analyses indicate that the sequences of IQGAP3-A24-9-955 (SEQ ID NO:2), IQGAP3-A24-9-1167 (SEQ ID NO:4), IQGAP3-A24-9-779 (SEQ ID NO:7), IQGAP3-A24-9-74 (SEQ ID NO: 21), IQGAP3-A24-9-26 (SEQ ID NO:25), IQGAP3-A24-9-137 (SEQ ID NO:29), IQGAP3-A24-9-63 (SEQ ID NO:32), IQGAP3-A24-10-1600 (SEQ ID NO:35), IQGAP3-A24-10-1507 (SEQ ID NO:37), IQGAP3-A24-10-139 (SEQ ID NO: 40), IQGAP3-A24-10-1097 (SEQ ID NO:49), IQGAP3-A24-10-345 (SEQ ID NO:53), IQGAP3-A24-10-1614 (SEQ ID NO:55), IQGAP3-A24-10-191 (SEQ ID NO:56), IQGAP3-A24-10-314 (SEQ ID NO:57), IQGAP3-A24-10-1363 (SEQ ID NO:62), IQGAP3-A24-10-1114 (SEQ ID NO:63) and IQGAP3-A24-10-1207 (SEQ ID NO: 67) are unique and thus, there is little possibility, to our best knowledge, that these molecules raise unintended immunologic response to some unrelated molecule.

In conclusion, novel HLA-A24 epitope peptides derived from IQGAP3 were identified and demonstrated to be applicable for cancer immunotherapy.

Prediction of HLA-A02 Binding Peptides Derived from IQGAP3

Table 2a and 2b show the HLA-A02 binding 9mer and 10mer peptides of IQGAP3 in the order of high binding affinity. A total of 84 peptides with potential HLA-A02 binding ability were selected and examined to determine the epitope peptides.

TABLE 2a
Table 2a; IILA-A02 binding 9mer
peptides derived from IQGAP3

| Start | Amino acid | Binding | SEQ ID |
|---|---|---|---|
| Position | sequence | Score | NO. |
| 1004 | YLLLQLFKT | 1691.953 | 69 |
| 1129 | FLLAITSSV | 1183.775 | 70 |
| 144 | ALSLFLFRL | 1082.903 | 71 |
| 1541 | QLLEKGVLV | 1055.104 | 72 |
| 783 | WLQYFKANL | 373.415 | 73 |
| 969 | YLAKLIFQM | 304.856 | 74 |
| 146 | SLFLFRLGL | 300.355 | 75 |
| 1055 | ILGKVIQDV | 271.948 | 76 |
| 813 | RLHYFQKNV | 264.298 | 77 |
| 962 | LLQTQPIYL | 199.738 | 78 |
| 1122 | LLAMTDKFL | 199.738 | 79 |
| 1486 | KLQATLQGL | 171.967 | 80 |
| 416 | SMYQLELAV | 160.742 | 81 |
| 1006 | LLQLFKTAL | 138.001 | 82 |
| 1365 | LLLSTKQLL | 134.369 | 83 |
| 1292 | LLLEHQDCI | 131.835 | 84 |
| 553 | GLDDVSLPV | 114.065 | 85 |
| 315 | ALQDPALAL | 87.586 | 86 |
| 1596 | LLQLQYEGV | 86.905 | 87 |
| 1051 | ALQEILGKV | 85.264 | 88 |
| 588 | WLEEIRQGV | 83.952 | 89 |
| 546 | ALLLPAAGL | 79.041 | 90 |
| 1364 | SLLLSTKQL | 79.041 | 91 |
| 1063 | VLEDKVLSV | 71.359 | 92 |
| 1598 | QLQYEGVAV | 69.552 | 93 |
| 376 | AMLHAVQRI | 64.121 | 94 |
| 985 | FMEAVIFSL | 60.592 | 95 |
| 405 | AQLPPVYPV | 60.011 | 96 |
| 663 | RPADTAFWV | 59.381 | 97 |
| 1005 | LLLQLFKTA | 59.373 | 98 |
| 1234 | VLNDYLEET | 58.537 | 99 |
| 1068 | VLSVHTDPV | 57.937 | 100 |
| 756 | FLRTWLPAV | 55.925 | 101 |
| 239 | NLREPLAAV | 49.847 | 102 |
| 153 | GLAPQLHDL | 49.134 | 103 |
| 934 | MVLDKQKGL | 48.205 | 104 |
| 911 | TLQEVVSHC | 46.848 | 105 |
| 896 | NIMDIKIGL | 44.559 | 106 |
| 1154 | TLAEKFPDA | 38.701 | 107 |
| 904 | LLVKNRITL | 36.316 | 108 |
| 989 | VIFSLYNYA | 35.448 | 109 |
| 194 | GILANELSV | 35.385 | 110 |

Start position indicates the number of amino acid residue from the N-terminus of IQGAP3.
Binding score is derived from “BIMAS”.

```
TABLE 2b
  Table 2b; HLA-A02 binding 10mer
  peptides derived from IQGAP3
```

| Start | Amino acid | Binding | SEQ ID |
|---|---|---|---|
| Position | sequence | Score | NO. |
| 961 | YLLQtQPIYL | 1999.734 | 111 |
| 725 | QLWKaNVGFV | 949.34 | 112 |
| 868 | FLAEaELLKL | 926.658 | 113 |
| 70 | KLGHcFAPSV | 925.042 | 114 |
| 1608 | KLFNkAKVNV | 900.698 | 115 |
| 802 | RMWAaRRQYL | 704.306 | 116 |
| 1005 | LLLQlFKTAL | 510.604 | 117 |
| 1121 | NLLAmTDKFL | 434.725 | 118 |
| 1013 | ALQEeIKSKV | 285.163 | 119 |
| 1124 | AMTDkFLLAI | 270.002 | 120 |
| 1174 | LLYYrFLNPA | 236.207 | 121 |
| 1122 | LLAMtDKFLL | 210.633 | 122 |
| 1004 | YLLLqLFKTA | 160.655 | 123 |
| 235 | ALLEnLREPL | 158.793 | 124 |
| 548 | LLPAaGLDDV | 133.255 | 125 |
| 1620 | LIFLlNKKFL | 101.617 | 126 |
| 109 | WLSAiAHIGL | 98.267 | 127 |
| 860 | LLNQsQQDFL | 97.872 | 128 |
| 1614 | KVNVnLLIFL | 82.759 | 129 |
| 903 | GLLVkNRITL | 79.041 | 130 |
| 1364 | SLLLsTKQLL | 79.041 | 131 |
| 501 | FLSWnDLQAT | 78.842 | 132 |
| 737 | LQARlRGFLV | 69.531 | 133 |
| 876 | KLQEeVVRKI | 68.867 | 134 |
| 438 | FVAVcMLSAV | 64.388 | 135 |
| 1154 | TLAEkFPDAT | 56.89 | 136 |
| 117 | GLPStFFPET | 53.803 | 137 |
| 1292 | LLLEhQDCIA | 52.529 | 138 |
| 953 | LEAYqHLFYL | 51.81 | 139 |
| 1590 | QLHYqDLLQL | 49.134 | 140 |
| 1424 | SLTAhSLLPL | 49.134 | 141 |
| 416 | SMYQlELAVL | 46.557 | 142 |
| 67 | LLAKlGHCFA | 46.451 | 143 |
| 1597 | LQLQyEGVAV | 44.356 | 144 |
| 1461 | GLVDeLAKDI | 42.774 | 145 |
| 1067 | KVLSvHTDPV | 38.617 | 146 |
| 921 | KLTKrNKEQL | 36.637 | 147 |
| 842 | ILVHaPHPPL | 36.316 | 148 |
| 1547 | VLVEiEDLPA | 34.627 | 149 |
| 897 | IMDkIGLLV | 34.158 | 150 |
| 1059 | VIQDvLEDKV | 32.662 | 151 |
| 1365 | LLLStKQLLA | 31.249 | 152 |

Start position indicates the number of amino acid residue from the N-terminus of IQGAP3.
Binding score is derived from “BIMAS”.

CTL Induction with the Predicted Peptides from IQGAP3 Restricted with HLA-A*0201

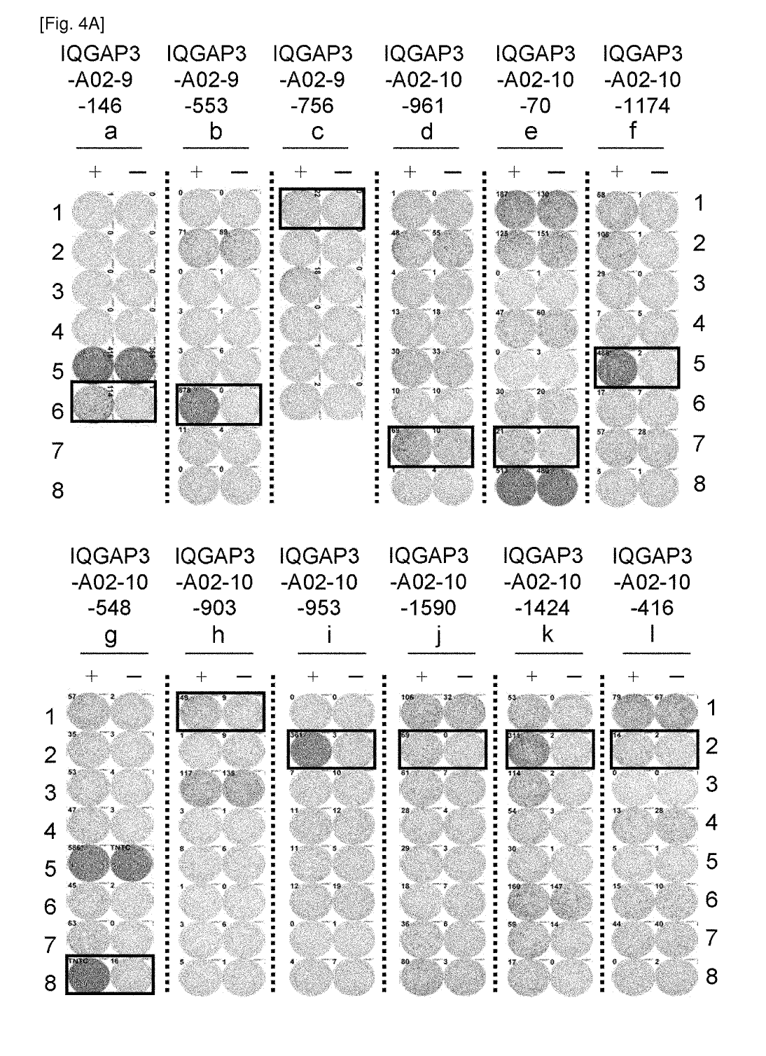

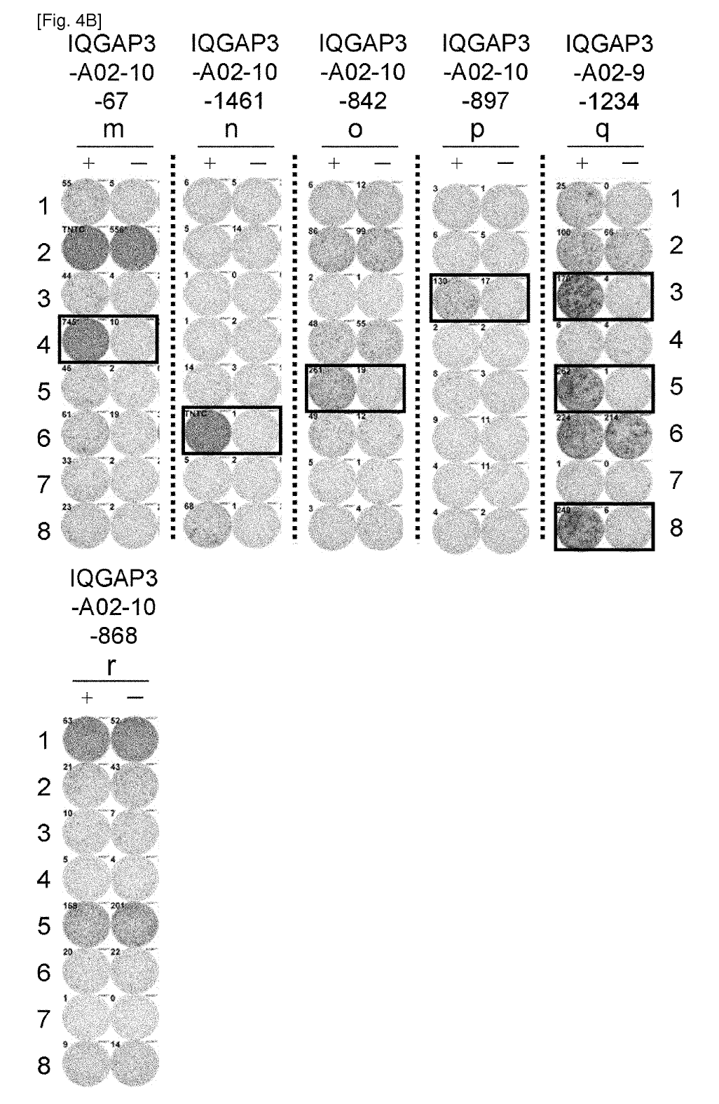

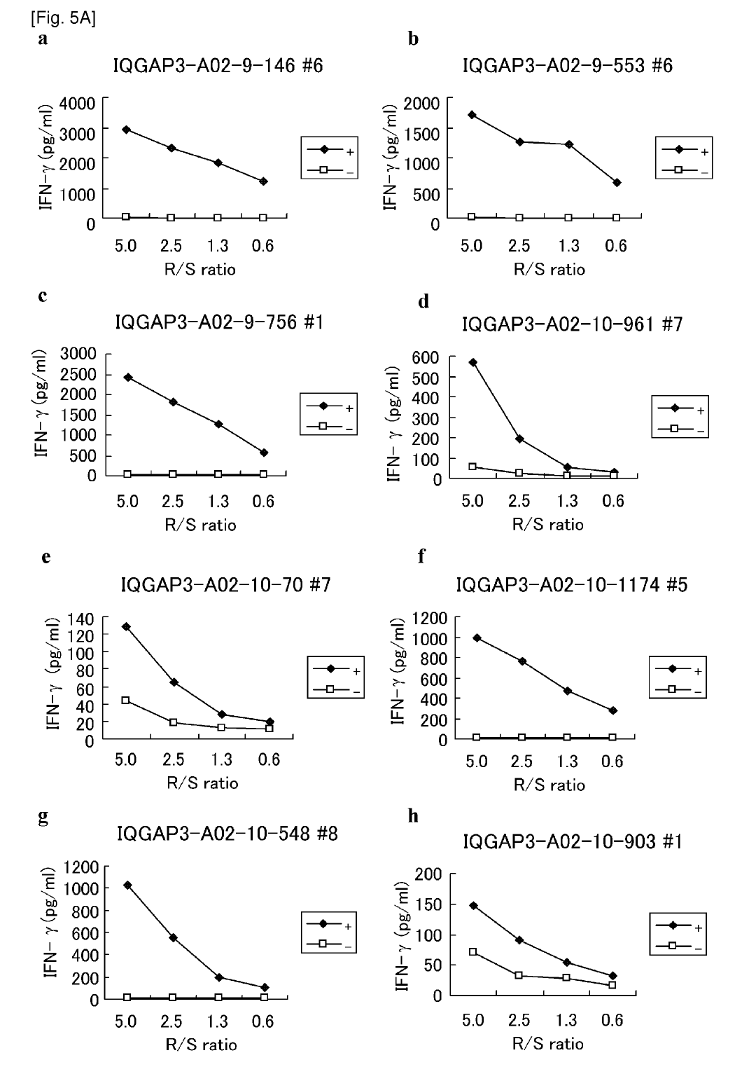

CTLs for those peptides derived from IQGAP3 were generated according to the protocols as described in “Materials and Methods”. Peptide specific CTL activity was determined by IFN-gamma ELISPOT assay (FIG. 4a-q). The results show that the well number #6 and 6 stimulated with IQGAP3-A02-9-146 (SEQ ID NO: 75) (a), #6 with IQGAP3-A02-9-553 (SEQ ID NO: 85) (b), #1 with IQGAP3-A02-9-756 (SEQ ID NO: 101) (c), #7 with IQGAP3-A02-10-961 (SEQ ID NO: 111) (d), #7 and 6 with IQGAP3-A02-10-70 (SEQ ID NO: 114) (e), #5 with IQGAP3-A02-10-1174 (SEQ ID NO: 121) (f), #8 with IQGAP3-A02-10-548 (SEQ ID NO: 125) (g), #1 with IQGAP3-A02-10-903 (SEQ ID NO: 130) (h), #2 with IQGAP3-A02-10-953 (SEQ ID NO: 139) (i), #2 with IQGAP3-A02-10-1590 (SEQ ID NO: 140) (j), #2 with IQGAP3-A02-10-1424 (SEQ ID NO: 141) (k), #2 with IQGAP3-A02-10-416 (SEQ ID NO: 142) (l), #4 with IQGAP3-A02-10-67 (SEQ ID NO: 143) (m), #6 with IQGAP3-A02-10-1461 (SEQ ID NO: 145) (n), #5 with IQGAP3-A02-10-842 (SEQ ID NO: 148) (o), #3 with IQGAP3-A02-10-897 (SEQ ID NO: 150) (p) and #5 with IQGAP3-A02-9-1234 (SEQ ID NO: 99) (q) demonstrated potent IFN-gamma production as compared to the control wells. On the other hand, no potent IFN-gamma production could be detected by stimulation with other peptides shown in Table 2, despite those peptides had possible binding activity with HLA-A*0201. As typical of negative data, no specific IFN-gamma production was observed from the CTL stimulated with IQGAP3-A02-10-868 (SEQ ID NO: 113) against peptide-pulsed target cells (r).

Establishment of CTL Lines and Clones Against IQGAP3 Specific Peptides

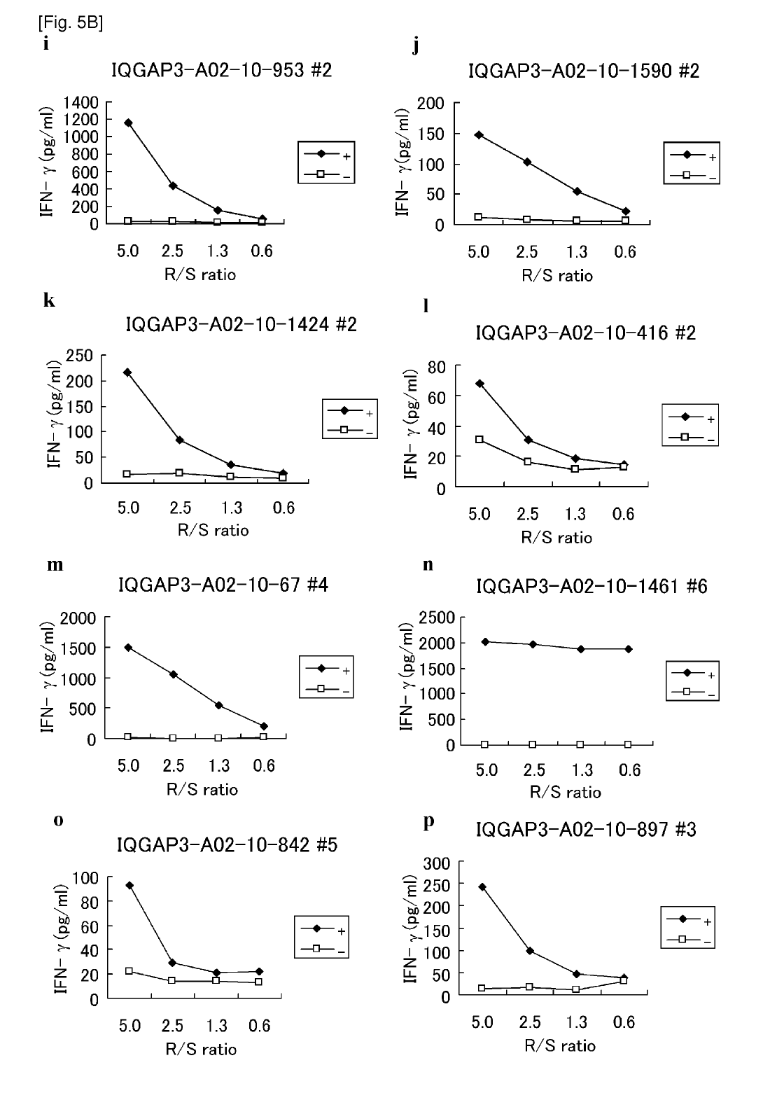

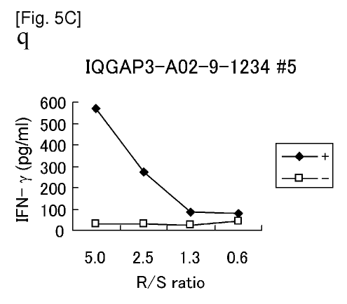

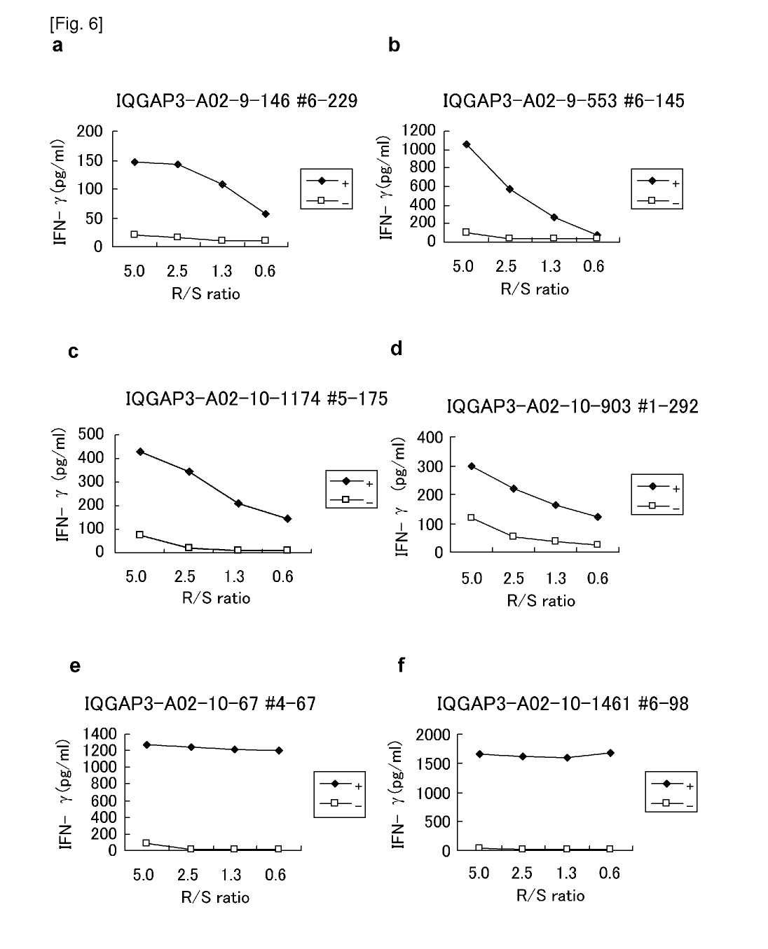

The cells that showed peptide specific CTL activity detected by IFN-gamma ELISPOT assay in the well number #6 and 6 stimulated with IQGAP3-A02-9-146 (SEQ ID NO: 75) (a), #6 with IQGAP3-A02-9-553 (SEQ ID NO: 85) (b), #1 with IQGAP3-A02-9-756 (SEQ ID NO: 101) (c), #7 with IQGAP3-A02-10-961 (SEQ ID NO: 111) (d), #7 and 6 with IQGAP3-A02-10-70 (SEQ ID NO: 114) (e), #5 with IQGAP3-A02-10-1174 (SEQ ID NO: 121) (f), #8 with IQGAP3-A02-10-548 (SEQ ID NO: 125) (g), #1 with IQGAP3-A02-10-903 (SEQ ID NO: 130) (h), #2 with IQGAP3-A02-10-953 (SEQ ID NO: 139) (i), #2 with IQGAP3-A02-10-1590 (SEQ ID NO: 140) (j), #2 with IQGAP3-A02-10-1424 (SEQ ID NO: 141) (k), #2 with IQGAP3-A02-10-416 (SEQ ID NO: 142) (l), #4 with IQGAP3-A02-10-67 (SEQ ID NO: 143) (m), #6 with IQGAP3-A02-10-1461 (SEQ ID NO: 145) (n), #5 with IQGAP3-A02-10-842 (SEQ ID NO: 148) (o), #3 with IQGAP3-A02-10-897 (SEQ ID NO: 150) (p) and #5 with IQGAP3-A02-9-1234 (SEQ ID NO: 99) were expanded and established CTL lines. CTL activity of those CTL lines was determined by IFN-gamma ELISA assay (FIG. 5a-q). It showed that all CTL lines demonstrated potent IFN-gamma production against the target cells pulsed with corresponding peptide as compared to target cells without peptide pulse. Furthermore, CTL clones were established by limiting dilution from CTL lines, and IFN-gamma production from CTL clones against target cells pulsed peptide were determined by IFN-gamma ELISA assay. Potent IFN-gamma productions were determined from CTL clones stimulated with IQGAP3-A02-9-146 (SEQ ID NO: 75) (a), IQGAP3-A02-9-553 (SEQ ID NO: 85) (b), IQGAP3-A02-10-1174 (SEQ ID NO: 121) (c), IQGAP3-A02-10-903 (SEQ ID NO: 130) (d), IQGAP3-A02-10-67 (SEQ ID NO: 143) (e) and IQGAP3-A02-10-1461 (SEQ ID NO: 145) (f) in FIG. 6.

Specific CTL Activity Against Target Cells Exogenously Expressing IQGAP3 and HLA-A*0201

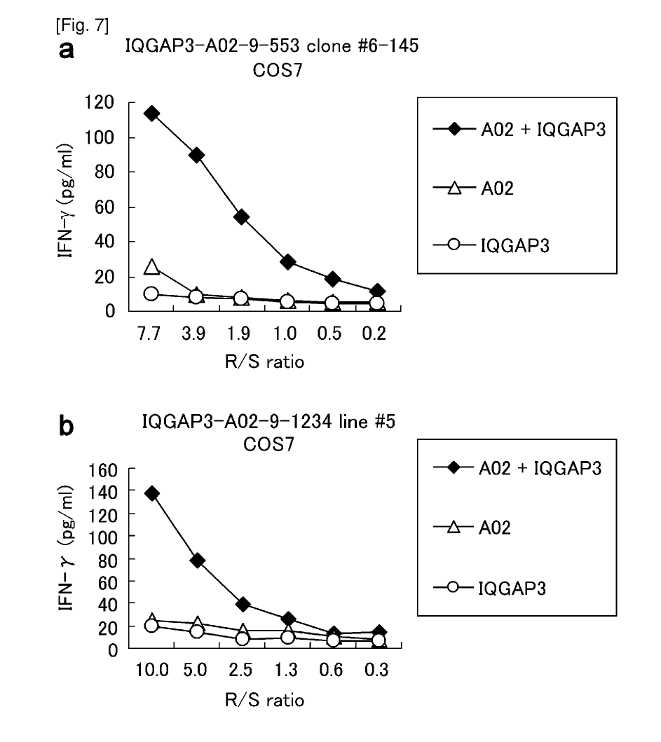

The established CTL clone raised against these peptides were examined for their ability to recognize target cells that endogenously express IQGAP3 and HLA-A*0201 molecule. Specific CTL activity against COS7 cells which transfected with both the full length of IQGAP3 and HLA-A*0201 molecule gene (a specific model for the target cells that endogenously express IQGAP3 and HLA-A*0201 gene) was tested using the CTL lines raised by corresponding peptide as the effecter cells. COS7 cells transfected with either full length of IQGAP3 genes or HLA-A*0201 were prepared as controls. In FIG. 7, the CTLs stimulated with IQGAP3-A02-9-553 (SEQ ID NO: 85) (a) and IQGAP3-A02-9-1234 (SEQ ID NO: 99) (b) showed potent CTL activity against COS7 cells expressing both IQGAP3 and HLA-A*0201. On the other hand, no significant specific CTL activity was detected against the controls. Thus, these data clearly demonstrate that peptides of IQGAP3-A02-9-553 (SEQ ID NO: 85) (a) and IQGAP3-A02-1234 (SEQ ID NO; 99) (b) are endogenously processed and expressed on the target cells with HLA-A*0201 molecule and were recognized by the CTLs. These results further indicate that IQGAP3-A02-9-553 (SEQ ID NO: 85) and IQGAP3-A02-9-1234 (SEQ ID NO: 99) may be applicable as a cancer vaccine for patients with IQGAP3 expressing tumors.

Homology Analysis of Antigen Peptides

The CTLs stimulated with IQGAP3-A02-9-146 (SEQ ID NO: 75), IQGAP3-A02-9-553 (SEQ ID NO: 85), IQGAP3-A02-9-1234 (SEQ ID NO: 99), IQGAP3-A02-9-756 (SEQ ID NO: 101), IQGAP3-A02-10-961 (SEQ ID NO: 111), IQGAP3-A02-10-70 (SEQ ID NO: 114), IQGAP3-A02-10-1174 (SEQ ID NO: 121), IQGAP3-A02-10-548 (SEQ ID NO: 125), IQGAP3-A02-10-903 (SEQ ID NO: 130), IQGAP3-A02-10-953 (SEQ ID NO: 139), IQGAP3-A02-10-1590 (SEQ ID NO: 140), IQGAP3-A02-10-1424 (SEQ ID NO: 141), IQGAP3-A02-10-416 (SEQ ID NO: 142), IQGAP3-A02-10-67 (SEQ ID NO: 143), IQGAP3-A02-10-1461 (SEQ ID NO: 145), IQGAP3-A02-10-842 (SEQ ID NO: 148) and IQGAP3-A02-10-897 (SEQ ID NO: 150) showed significant and specific CTL activity. This result may be due to the fact that the sequences of IQGAP3-A02-9-146 (SEQ ID NO: 75), IQGAP3-A02-9-553 (SEQ ID NO: 85), IQGAP3-A02-9-1234 (SEQ ID NO: 99), IQGAP3-A02-9-756 (SEQ ID NO: 101), IQGAP3-A02-10-961 (SEQ ID NO: 111), IQGAP3-A02-10-70 (SEQ ID NO: 114), IQGAP3-A02-10-1174 (SEQ ID NO: 121), IQGAP3-A02-10-548 (SEQ ID NO: 125), IQGAP3-A02-10-903 (SEQ ID NO: 130), IQGAP3-A02-10-953 (SEQ ID NO: 139), IQGAP3-A02-10-1590 (SEQ ID NO: 140), IQGAP3-A02-10-1424 (SEQ ID NO: 141), IQGAP3-A02-10-416 (SEQ ID NO: 142), IQGAP3-A02-10-67 (SEQ ID NO: 143), IQGAP3-A02-10-1461 (SEQ ID NO: 145), IQGAP3-A02-10-842 (SEQ ID NO: 148) and IQGAP3-A02-10-897 (SEQ ID NO: 150) are homologous to peptides derived from other molecules that are known to sensitize the human immune system. To exclude this possibility, homology analyses were performed for these peptide sequences using as queries the BLAST algorithm (http://www.ncbi.nlm.nih.gov/blast/blast.cgi) which revealed no sequence with significant homology. The results of homology analyses indicate that the sequences of IQGAP3-A02-9-146 (SEQ ID NO: 75), IQGAP3-A02-9-553 (SEQ ID NO: 85), IQGAP3-A02-9-1234 (SEQ ID NO: 99), IQGAP3-A02-9-756 (SEQ ID NO: 101), IQGAP3-A02-10-961 (SEQ ID NO: 111), IQGAP3-A02-10-70 (SEQ ID NO: 114), IQGAP3-A02-10-1174 (SEQ ID NO: 121), IQGAP3-A02-10-548 (SEQ ID NO: 125), IQGAP3-A02-10-903 (SEQ ID NO: 130), IQGAP3-A02-10-953 (SEQ ID NO: 139), IQGAP3-A02-10-1590 (SEQ ID NO: 140), IQGAP3-A02-10-1424 (SEQ ID NO: 141), IQGAP3-A02-10-416 (SEQ ID NO: 142), IQGAP3-A02-10-67 (SEQ ID NO: 143), IQGAP3-A02-10-1461 (SEQ ID NO:145), IQGAP3-A02-10-842 (SEQ ID NO: 148) and IQGAP3-A02-10-897 (SEQ ID NO: 150) are unique and thus, there is little possibility, to our best knowledge, that these molecules raise unintended immunologic response to some unrelated molecule.

In conclusion, a novel HLA-A02 epitope peptide derived from IQGAP3 has been established as a novel antigen and further demonstrated to be applicable for cancer immunotherapy.

### INDUSTRIAL APPLICABILITY

The present invention describes new TAAs, particularly those derived from IQGAP3 which induce potent and specific anti-tumor immune responses and have applicability to a wide array of cancer types. Such TAAs warrant further development as peptide vaccines against diseases associated with IQGAP3, e.g. cancer, more particularly, bladder, renal, esophageal, gastric, lung, breast, bladder and pancreatic cancer.

While the invention is herein described in detail and with reference to specific embodiments thereof, it is to be understood that the foregoing description is exemplary and explanatory in nature and is intended to illustrate the invention and its preferred embodiments. Through routine experimentation, one skilled in the art will readily recognize that various changes and modifications can be made therein without departing from the spirit and scope of the invention, the metes and bounds of which are defined by the appended claims.

1. An isolated nonapeptide or decapeptide having cytotoxic T cell inducibility, wherein said nonapeptide or decapeptide comprises an amino acid sequence selected from the amino acid sequence of SEQ ID NO: 154.

2. The nonapeptide or decapeptide of claim 1, wherein the peptide comprises an amino acid sequence selected from the group of: SEQ ID NO: 2, 4, 7, 21, 25, 29, 32, 35, 37, 40, 49, 53, 55, 56, 57, 62, 63, 67, 75, 85, 99, 101, 111, 114, 121, 125, 130, 139, 140, 141, 142, 143, 145, 148 and 150.

3. A peptide having cytotoxic T lymphocyte (CTL) inducibility, wherein the peptide comprises an amino acid sequence selected from the group of consisting of: (a) SEQ ID NO: 2, 4, 7, 21, 25, 29, 32, 35, 37, 40, 49, 53, 55, 56, 57, 62, 63, 67, 75, 85, 99, 101, 111, 114, 121, 125, 130, 139, 140, 141, 142, 143, 145, 148 and 150; or (b) SEQ ID NO: 2, 4, 7, 21, 25, 29, 32, 35, 37, 40, 49, 53, 55, 56, 57, 62, 63, 67, 75, 85, 99, 101, 111, 114, 121, 125, 130, 139, 140, 141, 142, 143, 145, 148 and 150, wherein 1, 2, or several amino acids are substituted, inserted, deleted or added.

4. The peptide of claim 3, wherein the peptide comprising the amino acid sequence selected from the group consisting of SEQ ID NOs: 2, 4, 7, 21, 25, 29, 32, 35, 37, 40, 49, 53, 55, 56, 57, 62, 63 and 67 has one or both of the following characteristics (a) the second amino acid from the N-terminus of the amino acid sequence of said SEQ ID NOs is or is modified to be an amino acid selected from the group consisting of phenylalanine, tyrosine, methionine and tryptophan, and (b) the C-terminal amino acid of the amino acid sequence of said SEQ ID NOs is or is modified to be an amino acid selected from the group consisting of phenylalanine, leucine, isoleucine, tryptophan and methionine.

5. The peptide of claim 3, wherein the peptide comprising the amino acid sequence selected from the group consisting of SEQ ID NOs: 75, 85, 99, 101, 111, 114, 121, 125, 130, 139, 140, 141, 142, 143, 145, 148 and 150 has one or both of the following characteristics: (a) the second amino acid from the N-terminus of the amino acid sequence of said SEQ ID NOs is or is modified to be an amino acid selected from the group consisting of leucine or methionine, and (b) the C-terminal amino acid of the amino acid sequence of said SEQ ID NOs is or is modified to be an amino acid selected from the group consisting of valine or leucine.

6. A pharmaceutical composition comprising one or more peptides of claim 1, or a polynucleotide encoding such a peptide, in combination with a pharmacologically acceptable carrier formulated for a purpose selected from the group consisting of:
(i) treatment of a tumor,
(ii) prophylaxis of a tumor,
(iii) preventing postoperative recurrence of a tumor, and
(iv) combinations thereof.

7. The pharmaceutical composition of claim 6, formulated for the administration to a subject whose HLA antigen is HLA-A24 or HLA-A02.

8. The pharmaceutical composition of claim 7, formulated for the treatment of cancer.

9. The pharmaceutical composition of claim 8, wherein said composition comprises a vaccine.

10. An exosome that presents on its surface a complex comprising a peptide as set forth in claim 1, in combination with an HLA antigen.

11. The exosome of claim 10, wherein the HLA antigen is HLA-A24.

12. The exosome of claim 10, wherein the HLA antigen is HLA-A2402.

13. The exosome of claim 10, wherein the HLA antigen is HLA-A02.

14. The exosome of claim 10, wherein the HLA antigen is HLA-A0201.

15. A method for inducing an antigen-presenting cell with high CTL inducibility by using a peptide as set forth in claim 1.

16. A method for inducing CTL by using a peptide as set forth in claim 1.

17. The method for inducing an antigen-presenting cell with high CTL inducibility of claim 15, wherein said method comprises the step of introducing a gene that comprises a polynucleotide encoding an isolated nonapeptide or decapeptide having cytotoxic T cell inducibility, wherein said nonapeptide or decapeptide comprises an amino acid sequence selected from the amino acid sequence of SEQ ID NO: 154, into an antigen-presenting cell.

18. An isolated cytotoxic T cell which targets any of the peptides of claim 1.

19. An isolated cytotoxic T cell that is induced by using a peptide as set forth in claim 1.

20. An isolated antigen-presenting cell that presents on its surface a complex of an HLA antigen and a peptide as set forth in claim 1.

21. The antigen-presenting cell of claim 20, wherein said cell is induced by a method for inducing an antigen-presenting cell with high CTL inducibility by using a nonapeptide or decapeptide having cytotoxic T cell inducibility, wherein said nonapeptide or decapeptide comprises an amino acid sequence selected from the amino acid sequence of SEQ ID NO: 154.

22. A method of inducing an immune response against a cancer in a subject, said method comprising the step of administering to said subject a vaccine comprising a peptide as set forth in claim 1, an immunologically active fragment thereof, or a polynucleotide encoding such a peptide or fragment.

23. A pharmaceutical composition comprising one or more peptides of claim 3, or a polynucleotide encoding such a peptide, in combination with a pharmacologically acceptable carrier formulated for a purpose selected from the group consisting of:
(i) treatment of a tumor,
(ii) prophylaxis of a tumor,
(iii) preventing postoperative recurrence of a tumor, and
(iv) combinations thereof.

24. The pharmaceutical composition of claim 23, formulated for the administration to a subject whose HLA antigen is HLA-A24 or HLA-A02.

25. The pharmaceutical composition of claim 24, formulated for the treatment of cancer.

26. The pharmaceutical composition of claim 25, wherein said composition comprises a vaccine.

27. An exosome that presents on its surface a complex comprising a peptide as set forth in claim 3, in combination with an HLA antigen.

28. The exosome of claim 27, wherein the HLA antigen is HLA-A24.

29. The exosome of claim 27, wherein the HLA antigen is HLA-A2402.

30. The exosome of claim 27, wherein the HLA antigen is HLA-A02.

31. The exosome of claim 27, wherein the HLA antigen is HLA-A0201.

32. A method for inducing an antigen-presenting cell with high CTL inducibility by using a peptide as set forth in claim 3.

33. A method for inducing CTL by using a peptide as set forth in claim 3.

34. The method for inducing an antigen-presenting cell with high CTL inducibility of claim 32, wherein said method comprises the step of introducing a gene that comprises a polynucleotide encoding a peptide having cytotoxic T lymphocyte (CTL) inducibility, wherein the peptide comprises an amino acid sequence selected from the group of consisting of: (a) SEQ ID NO: 2, 4, 7, 21, 25, 29, 32, 35, 37, 40, 49, 53, 55, 56, 57, 62, 63, 67, 75, 85, 99, 101, 111, 114, 121, 125, 130, 139, 140, 141, 142, 143, 145, 148 and 150; or (b) SEQ ID NO: 2, 4, 7, 21, 25, 29, 32, 35, 37, 40, 49, 53, 55, 56, 57, 62, 63, 67, 75, 85, 99, 101, 111, 114, 121, 125, 130, 139, 140, 141, 142, 143, 145, 148 and 150, wherein 1, 2, or several amino acids are substituted, inserted, deleted or added, into an antigen-presenting cell.

35. An isolated cytotoxic T cell which targets any of the peptides of claim 3.

36. An isolated cytotoxic T cell that is induced by using a peptide as set forth in claim 3.

37. An isolated antigen-presenting cell that presents on its surface a complex of an HLA antigen and a peptide as set forth in claim 3.

38. The antigen-presenting cell of claim 37, wherein said cell is induced by a method for inducing an antigen-presenting cell with high CTL inducibility by using a peptide having cytotoxic T lymphocyte (CTL) inducibility, wherein the peptide comprises an amino acid sequence selected from the group of consisting of: (a) SEQ ID NO: 2, 4, 7, 21, 25, 29, 32, 35, 37, 40, 49, 53, 55, 56, 57, 62, 63, 67, 75, 85, 99, 101, 111, 114, 121, 125, 130, 139, 140, 141, 142, 143, 145, 148 and 150; or (b) SEQ ID NO: 2, 4, 7, 21, 25, 29, 32, 35, 37, 40, 49, 53, 55, 56, 57, 62, 63, 67, 75, 85, 99, 101, 111, 114, 121, 125, 130, 139, 140, 141, 142, 143, 145, 148 and 150, wherein 1, 2, or several amino acids are substituted, inserted, deleted or added.

39. A method of inducing an immune response against a cancer in a subject, said method comprising the step of administering to said subject a vaccine comprising a peptide as set forth in claim 3, an immunologically active fragment thereof, or a polynucleotide encoding such a peptide or fragment.


SEQUENCE LISTING (154 sequences)

SEQ ID NO: 1 - protein, 9 aa, Artificial
  An artificially synthesized peptide sequence
RYFDALLKL

SEQ ID NO: 2 - protein, 9 aa, Artificial
  An artificially synthesized peptide sequence
AYQHLFYLL

SEQ ID NO: 3 - protein, 9 aa, Artificial
  An artificially synthesized peptide sequence
GYQGLVDEL

SEQ ID NO: 4 - protein, 9 aa, Artificial
  An artificially synthesized peptide sequence
VYKVVGNLL

SEQ ID NO: 5 - protein, 9 aa, Artificial
  An artificially synthesized peptide sequence
RYQATGLHF

SEQ ID NO: 6 - protein, 9 aa, Artificial
  An artificially synthesized peptide sequence
MYQLELAVL

SEQ ID NO: 7 - protein, 9 aa, Artificial
  An artificially synthesized peptide sequence
IYLEWLQYF

SEQ ID NO: 8 - protein, 9 aa, Artificial
  An artificially synthesized peptide sequence
VYCIHALSL

SEQ ID NO: 9 - protein, 9 aa, Artificial
  An artificially synthesized peptide sequence
KYGLQLPAF

SEQ ID NO: 10 - protein, 9 aa, Artificial
  An artificially synthesized peptide sequence
GYRQRKIYL

SEQ ID NO: 11 - protein, 9 aa, Artificial
  An artificially synthesized peptide sequence
QYLRRLHYF

SEQ ID NO: 12 - protein, 9 aa, Artificial
  An artificially synthesized peptide sequence
AYYFHLQTF

SEQ ID NO: 13 - protein, 9 aa, Artificial
  An artificially synthesized peptide sequence
FYLLQTQPI

SEQ ID NO: 14 - protein, 9 aa, Artificial
  An artificially synthesized peptide sequence
RFQLHYQDL

SEQ ID NO: 15 - protein, 9 aa, Artificial
  An artificially synthesized peptide sequence
KFEVNAKFL

SEQ ID NO: 16 - protein, 9 aa, Artificial
  An artificially synthesized peptide sequence
KFAEHSHFL

SEQ ID NO: 17 - protein, 9 aa, Artificial
  An artificially synthesized peptide sequence
IFLLNKKFL

SEQ ID NO: 18 - protein, 9 aa, Artificial
  An artificially synthesized peptide sequence
DFLAEAELL

SEQ ID NO: 19 - protein, 9 aa, Artificial
  An artificially synthesized peptide sequence
AFSKIGGIL

SEQ ID NO: 20 - protein, 9 aa, Artificial
  An artificially synthesized peptide sequence
AFSGQSQHL

SEQ ID NO: 21 - protein, 9 aa, Artificial
  An artificially synthesized peptide sequence
CFAPSVVPL

SEQ ID NO: 22 - protein, 9 aa, Artificial
  An artificially synthesized peptide sequence
RYVAKVLKA

SEQ ID NO: 23 - protein, 9 aa, Artificial
  An artificially synthesized peptide sequence
KAQDDYRIL

SEQ ID NO: 24 - protein, 9 aa, Artificial
  An artificially synthesized peptide sequence
KLQATLQGL

SEQ ID NO: 25 - protein, 9 aa, Artificial
  An artificially synthesized peptide sequence
RQNVAYQYL

SEQ ID NO: 26 - protein, 9 aa, Artificial
  An artificially synthesized peptide sequence
RVLRNLRRL

SEQ ID NO: 27 - protein, 9 aa, Artificial
  An artificially synthesized peptide sequence
RSLTAHSLL

SEQ ID NO: 28 - protein, 9 aa, Artificial
  An artificially synthesized peptide sequence
RYHLLLVAA

SEQ ID NO: 29 - protein, 9 aa, Artificial
  An artificially synthesized peptide sequence
RVVYCIHAL

SEQ ID NO: 30 - protein, 9 aa, Artificial
  An artificially synthesized peptide sequence
RNLRRLEAL

SEQ ID NO: 31 - protein, 9 aa, Artificial
  An artificially synthesized peptide sequence
KQRRVLRNL

SEQ ID NO: 32 - protein, 9 aa, Artificial
  An artificially synthesized peptide sequence
RNGVLLAKL

SEQ ID NO: 33 - protein, 9 aa, Artificial
  An artificially synthesized peptide sequence
VYITVGELV

SEQ ID NO: 34 - protein, 9 aa, Artificial
  An artificially synthesized peptide sequence
NIMDIKIGL

SEQ ID NO: 35 - protein, 10 aa, Artificial
  An artificially synthesized peptide sequence
QYEGVAVMKL

SEQ ID NO: 36 - protein, 10 aa, Artificial
  An artificially synthesized peptide sequence
QYIRACLDHL

SEQ ID NO: 37 - protein, 10 aa, Artificial
  An artificially synthesized peptide sequence
YYSQYIRACL

SEQ ID NO: 38 - protein, 10 aa, Artificial
  An artificially synthesized peptide sequence
DYLEETHLKF

SEQ ID NO: 39 - protein, 10 aa, Artificial
  An artificially synthesized peptide sequence
KFMEAVIFSL

SEQ ID NO: 40 - protein, 10 aa, Artificial
  An artificially synthesized peptide sequence
VYCIHALSLF

SEQ ID NO: 41 - protein, 10 aa, Artificial
  An artificially synthesized peptide sequence
RFQLHYQDLL

SEQ ID NO: 42 - protein, 10 aa, Artificial
  An artificially synthesized peptide sequence
HYFQKNVNSI

SEQ ID NO: 43 - protein, 10 aa, Artificial
  An artificially synthesized peptide sequence
QYFKANLDAI

SEQ ID NO: 44 - protein, 10 aa, Artificial
  An artificially synthesized peptide sequence
IYLAKLIFQM

SEQ ID NO: 45 - protein, 10 aa, Artificial
  An artificially synthesized peptide sequence
GYQRALESAM

SEQ ID NO: 46 - protein, 10 aa, Artificial
  An artificially synthesized peptide sequence
AYERLTAEEM

SEQ ID NO: 47 - protein, 10 aa, Artificial
  An artificially synthesized peptide sequence
GFVIQLQARL

SEQ ID NO: 48 - protein, 10 aa, Artificial
  An artificially synthesized peptide sequence
KFLGVDMERF

SEQ ID NO: 49 - protein, 10 aa, Artificial
  An artificially synthesized peptide sequence
PYDVTPEQAL

SEQ ID NO: 50 - protein, 10 aa, Artificial
  An artificially synthesized peptide sequence
DFADWYLEQL

SEQ ID NO: 51 - protein, 10 aa, Artificial
  An artificially synthesized peptide sequence
RYVAKVLKAT

SEQ ID NO: 52 - protein, 10 aa, Artificial
  An artificially synthesized peptide sequence
RSNQQLEQDL

SEQ ID NO: 53 - protein, 10 aa, Artificial
  An artificially synthesized peptide sequence
KAQELGLVEL

SEQ ID NO: 54 - protein, 10 aa, Artificial
  An artificially synthesized peptide sequence
RGQSALQEIL

SEQ ID NO: 55 - protein, 10 aa, Artificial
  An artificially synthesized peptide sequence
KVNVNLLIFL

SEQ ID NO: 56 - protein, 10 aa, Artificial
  An artificially synthesized peptide sequence
KIGGILANEL

SEQ ID NO: 57 - protein, 10 aa, Artificial
  An artificially synthesized peptide sequence
KALQDPALAL

SEQ ID NO: 58 - protein, 10 aa, Artificial
  An artificially synthesized peptide sequence
KGVLVEIEDL

SEQ ID NO: 59 - protein, 10 aa, Artificial
  An artificially synthesized peptide sequence
RVLRNPAVAL

SEQ ID NO: 60 - protein, 10 aa, Artificial
  An artificially synthesized peptide sequence
KYGLQLPAFS

SEQ ID NO: 61 - protein, 10 aa, Artificial
  An artificially synthesized peptide sequence
KANVGFVIQL

SEQ ID NO: 62 - protein, 10 aa, Artificial
  An artificially synthesized peptide sequence
RSLLLSTKQL

SEQ ID NO: 63 - protein, 10 aa, Artificial
  An artificially synthesized peptide sequence
RLDIALRNLL

SEQ ID NO: 64 - protein, 10 aa, Artificial
  An artificially synthesized peptide sequence
HYQDLLQLQY

SEQ ID NO: 65 - protein, 10 aa, Artificial
  An artificially synthesized peptide sequence
GYQGLVDELA

SEQ ID NO: 66 - protein, 10 aa, Artificial
  An artificially synthesized peptide sequence
GALEVVDDAL

SEQ ID NO: 67 - protein, 10 aa, Artificial
  An artificially synthesized peptide sequence
HALGAVAQLL

SEQ ID NO: 68 - protein, 10 aa, Artificial
  An artificially synthesized peptide sequence
HFRHTDNINF

SEQ ID NO: 69 - protein, 9 aa, Artificial
  An artificially synthesized peptide sequence
YLLLQLFKT

SEQ ID NO: 70 - protein, 9 aa, Artificial
  An artificially synthesized peptide sequence
FLLAITSSV

SEQ ID NO: 71 - protein, 9 aa, Artificial
  An artificially synthesized peptide sequence
ALSLFLFRL

SEQ ID NO: 72 - protein, 9 aa, Artificial
  An artificially synthesized peptide sequence
QLLEKGVLV

SEQ ID NO: 73 - protein, 9 aa, Artificial
  An artificially synthesized peptide sequence
WLQYFKANL

SEQ ID NO: 74 - protein, 9 aa, Artificial
  An artificially synthesized peptide sequence
YLAKLIFQM

SEQ ID NO: 75 - protein, 9 aa, Artificial
  An artificially synthesized peptide sequence
SLFLFRLGL

SEQ ID NO: 76 - protein, 9 aa, Artificial
  An artificially synthesized peptide sequence
ILGKVIQDV

SEQ ID NO: 77 - protein, 9 aa, Artificial
  An artificially synthesized peptide sequence
RLHYFQKNV

SEQ ID NO: 78 - protein, 9 aa, Artificial
  An artificially synthesized peptide sequence
LLQTQPIYL

SEQ ID NO: 79 - protein, 9 aa, Artificial
  An artificially synthesized peptide sequence
LLAMTDKFL

SEQ ID NO: 80 - protein, 9 aa, Artificial
  An artificially synthesized peptide sequence
KLQATLQGL

SEQ ID NO: 81 - protein, 9 aa, Artificial
  An artificially synthesized peptide sequence
SMYQLELAV

SEQ ID NO: 82 - protein, 9 aa, Artificial
  An artificially synthesized peptide sequence
LLQLFKTAL

SEQ ID NO: 83 - protein, 9 aa, Artificial
  An artificially synthesized peptide sequence
LLLSTKQLL

SEQ ID NO: 84 - protein, 9 aa, Artificial
  An artificially synthesized peptide sequence
LLLEHQDCI

SEQ ID NO: 85 - protein, 9 aa, Artificial
  An artificially synthesized peptide sequence
GLDDVSLPV

SEQ ID NO: 86 - protein, 9 aa, Artificial
  An artificially synthesized peptide sequence
ALQDPALAL

SEQ ID NO: 87 - protein, 9 aa, Artificial
  An artificially synthesized peptide sequence
LLQLQYEGV

SEQ ID NO: 88 - protein, 9 aa, Artificial
  An artificially synthesized peptide sequence
ALQEILGKV

SEQ ID NO: 89 - protein, 9 aa, Artificial
  An artificially synthesized peptide sequence
WLEEIRQGV

SEQ ID NO: 90 - protein, 9 aa, Artificial
  An artificially synthesized peptide sequence
ALLLPAAGL

SEQ ID NO: 91 - protein, 9 aa, Artificial
  An artificially synthesized peptide sequence
SLLLSTKQL

SEQ ID NO: 92 - protein, 9 aa, Artificial
  An artificially synthesized peptide sequence
VLEDKVLSV

SEQ ID NO: 93 - protein, 9 aa, Artificial
  An artificially synthesized peptide sequence
QLQYEGVAV

SEQ ID NO: 94 - protein, 9 aa, Artificial
  An artificially synthesized peptide sequence
AMLHAVQRI

SEQ ID NO: 95 - protein, 9 aa, Artificial
  An artificially synthesized peptide sequence
FMEAVIFSL

SEQ ID NO: 96 - protein, 9 aa, Artificial
  An artificially synthesized peptide sequence
AQLPPVYPV

SEQ ID NO: 97 - protein, 9 aa, Artificial
  An artificially synthesized peptide sequence
RPADTAFWV

SEQ ID NO: 98 - protein, 9 aa, Artificial
  An artificially synthesized peptide sequence
LLLQLFKTA

SEQ ID NO: 99 - protein, 9 aa, Artificial
  An artificially synthesized peptide sequence
VLNDYLEET

SEQ ID NO: 100 - protein, 9 aa, Artificial
  An artificially synthesized peptide sequence
VLSVHTDPV

SEQ ID NO: 101 - protein, 9 aa, Artificial
  An artificially synthesized peptide sequence
FLRTWLPAV

SEQ ID NO: 102 - protein, 9 aa, Artificial
  An artificially synthesized peptide sequence
NLREPLAAV

SEQ ID NO: 103 - protein, 9 aa, Artificial
  An artificially synthesized peptide sequence
GLAPQIHDL

SEQ ID NO: 104 - protein, 9 aa, Artificial
  An artificially synthesized peptide sequence
MVLDKQKGL

SEQ ID NO: 105 - protein, 9 aa, Artificial
  An artificially synthesized peptide sequence
TLQEVVSHC

SEQ ID NO: 106 - protein, 9 aa, Artificial
  An artificially synthesized peptide sequence
NIMDIKIGL

SEQ ID NO: 107 - protein, 9 aa, Artificial
  An artificially synthesized peptide sequence
TLAEKFPDA

SEQ ID NO: 108 - protein, 9 aa, Artificial
  An artificially synthesized peptide sequence
LLVKNRITL

SEQ ID NO: 109 - protein, 9 aa, Artificial
  An artificially synthesized peptide sequence
VIFSLYNYA

SEQ ID NO: 110 - protein, 9 aa, Artificial
  An artificially synthesized peptide sequence
GILANELSV

SEQ ID NO: 111 - protein, 10 aa, Artificial
  An artificially synthesized peptide sequence
YLLQTQPIYL

SEQ ID NO: 112 - protein, 10 aa, Artificial
  An artificially synthesized peptide sequence
QLWKANVGFV

SEQ ID NO: 113 - protein, 10 aa, Artificial
  An artificially synthesized peptide sequence
FLAEAELLKL

SEQ ID NO: 114 - protein, 10 aa, Artificial
  An artificially synthesized peptide sequence
KLGHCFAPSV

SEQ ID NO: 115 - protein, 10 aa, Artificial
  An artificially synthesized peptide sequence
KLFNKAKVNV

SEQ ID NO: 116 - protein, 10 aa, Artificial
  An artificially synthesized peptide sequence
RMWAARRQYL

SEQ ID NO: 117 - protein, 10 aa, Artificial
  An artificially synthesized peptide sequence
LLLQLFKTAL

SEQ ID NO: 118 - protein, 10 aa, Artificial
  An artificially synthesized peptide sequence
NLLAMTDKFL

SEQ ID NO: 119 - protein, 10 aa, Artificial
  An artificially synthesized peptide sequence
ALQEEIKSKV

SEQ ID NO: 120 - protein, 10 aa, Artificial
  An artificially synthesized peptide sequence
AMTDKFLLAI

SEQ ID NO: 121 - protein, 10 aa, Artificial
  An artificially synthesized peptide sequence
LLYYRFLNPA

SEQ ID NO: 122 - protein, 10 aa, Artificial
  An artificially synthesized peptide sequence
LLAMTDKFLL

SEQ ID NO: 123 - protein, 10 aa, Artificial
  An artificially synthesized peptide sequence
YLLLQLFKTA

SEQ ID NO: 124 - protein, 10 aa, Artificial
  An artificially synthesized peptide sequence
ALLENLREPL

SEQ ID NO: 125 - protein, 10 aa, Artificial
  An artificially synthesized peptide sequence
LLPAAGLDDV

SEQ ID NO: 126 - protein, 10 aa, Artificial
  An artificially synthesized peptide sequence
LIFLLNKKFL

SEQ ID NO: 127 - protein, 10 aa, Artificial
  An artificially synthesized peptide sequence
WLSAIAHIGL

SEQ ID NO: 128 - protein, 10 aa, Artificial
  An artificially synthesized peptide sequence
LLNQSQQDFL

SEQ ID NO: 129 - protein, 10 aa, Artificial
  An artificially synthesized peptide sequence
KVNVNLLIFL

SEQ ID NO: 130 - protein, 10 aa, Artificial
  An artificially synthesized peptide sequence
GLLVKNRITL

SEQ ID NO: 131 - protein, 10 aa, Artificial
  An artificially synthesized peptide sequence
SLLLSTKQLL

SEQ ID NO: 132 - protein, 10 aa, Artificial
  An artificially synthesized peptide sequence
FLSWNDLQAT

SEQ ID NO: 133 - protein, 10 aa, Artificial
  An artificially synthesized peptide sequence
LQARLRGFLV

SEQ ID NO: 134 - protein, 10 aa, Artificial
  An artificially synthesized peptide sequence
KLQEEVVRKI

SEQ ID NO: 135 - protein, 10 aa, Artificial
  An artificially synthesized peptide sequence
FVAVEMLSAV

SEQ ID NO: 136 - protein, 10 aa, Artificial
  An artificially synthesized peptide sequence
TLAEKFPDAT

SEQ ID NO: 137 - protein, 10 aa, Artificial
  An artificially synthesized peptide sequence
GLPSTFFPET

SEQ ID NO: 138 - protein, 10 aa, Artificial
  An artificially synthesized peptide sequence
LLLEHQDCIA

SEQ ID NO: 139 - protein, 10 aa, Artificial
  An artificially synthesized peptide sequence
LEAYQHLFYL

SEQ ID NO: 140 - protein, 10 aa, Artificial
  An artificially synthesized peptide sequence
QLHYQDLLQL

SEQ ID NO: 141 - protein, 10 aa, Artificial
  An artificially synthesized peptide sequence
SLTAHSLLPL

SEQ ID NO: 142 - protein, 10 aa, Artificial
  An artificially synthesized peptide sequence
SMYQLELAVL

SEQ ID NO: 143 - protein, 10 aa, Artificial
  An artificially synthesized peptide sequence
LLAKLGHCFA

SEQ ID NO: 144 - protein, 10 aa, Artificial
  An artificially synthesized peptide sequence
LQLQYEGVAV

SEQ ID NO: 145 - protein, 10 aa, Artificial
  An artificially synthesized peptide sequence
GLVDELAKDI

SEQ ID NO: 146 - protein, 10 aa, Artificial
  An artificially synthesized peptide sequence
KVLSVHTDPV

SEQ ID NO: 147 - protein, 10 aa, Artificial
  An artificially synthesized peptide sequence
KLTKRNKEQL

SEQ ID NO: 148 - protein, 10 aa, Artificial
  An artificially synthesized peptide sequence
ILVHAPHPPL

SEQ ID NO: 149 - protein, 10 aa, Artificial
  An artificially synthesized peptide sequence
VLVEIEDLPA

SEQ ID NO: 150 - protein, 10 aa, Artificial
  An artificially synthesized peptide sequence
IMDIKIGLLV

SEQ ID NO: 151 - protein, 10 aa, Artificial
  An artificially synthesized peptide sequence
VIQDVLEDKV

SEQ ID NO: 152 - protein, 10 aa, Artificial
  An artificially synthesized peptide sequence
LLLSTKQLLA

SEQ ID NO: 153 - DNA, 6069 bp, Homo sapiens
  CDS (76)..(4971)
gtcctgtctggcggtgccgacggtgaggggcggtggcccaacggcgggagattcaaacctggaagaaggaggaacatggagaggagagcagcgggcccaggctgggcagcctatgaacgcctcacagctgaggagatggatgagcagaggcggcagaatgttgcctatcagtacctgtgccggctggaggaggccaagcgctggatggaggcctgcctgaaggaggagcttccttccccggtggagctggaggagagccttcggaatggagtgctgctggccaagctaggccactgttttgcaccctccgtggttcccttgaagaagatctacgatgtggagcagctgcggtaccaggcaactggcttacatttccgtcacacagacaacatcaacttttggctatctgcaatagcccacatcggtctgccttcgaccttcttcccagagaccacggacatctatgacaaaaagaacatgccccgggtagtctactgcatccatgctctcagtctcttcctcttccggctgggattggcccctcagatacatgatctatacgggaaagtgaaattcacagctgaggaactcagcaacatggcgtccgaactggccaaatatggcctccagctgcctgccttcagcaagatcgggggcatcttggccaatgagctctcggtggatgaggctgcagtccatgcagctgttcttgccatcaatgaagcagtggagcgaggggtggtggaggacaccctggctgccttgcagaatcccagtgctcttctggagaatctccgagagcctctggcagccgtctaccaagagatgctggcccaggccaagatggagaaggcagccaatgccaggaaccatgatgacagagaaagccaggacatctatgaccactacctaactcaggctgaaatccagggcaatatcaaccatgtcaacgtccatggggctctagaagttgttgatgatgccctggaaagacagagccctgaagccttgctcaaggcccttcaagaccctgccctggccctgcgaggggtgaggagagactttgctgactggtacctggagcagctgaactcagacagagagcagaaggcacaggagctgggcctggtggagcttctggaaaaggaggaagtccaggctggtgtggctgcagccaacacaaagggtgatcaggaacaagccatgctccacgctgtgcagcggatcaacaaagccatccggaggggagtggcggctgacactgtgaaggagctgatgtgccctgaggcccagctgcctccagtgtaccctgttgcatcgtctatgtaccagctggagctggcagtgctccagcagcagcagggggagcttggccaggaggagctcttcgtggctgtggagatgctctcagctgtggtcctgattaaccgggccctggaggcccgggatgccagtggcttctggagcagcctggtgaaccctgccacaggcctggctgaggtggaaggagaaaatgcccagcgttacttcgatgccctgctgaaattgcgacaggagcgtgggatgggtgaggacttcctgagctggaatgacctgcaggccaccgtgagccaggtcaatgcacagacccaggaagagactgaccgggtccttgcagtcagcctcatcaatgaggctctggacaaaggcagccctgagaagactctgtctgccctactgcttcctgcagctggcctagatgatgtcagcctccctgtcgcccctcggtaccatctcctccttgtggcagccaaaaggcagaaggcccaggtgacaggggatcctggagctgtgctgtggcttgaggagatccgccagggagtggtcagagccaaccaggacactaatacagctcagagaatggctcttggtgtggctgccatcaatcaagccatcaaggagggcaaggcagcccagactgagcgggtgttgaggaaccccgcagtggcccttcgaggggtagttcccgactgtgccaacggctaccagcgagccctggaaagtgccatggcaaagaaacagcgtccagcagacacagctttctgggttcaacatgacatgaaggatggcactgcctactacttccatctgcagaccttccaggggatctgggagcaacctcctggctgccccctcaacacctctcacctgacccgggaggagatccagtcagctgtcaccaaggtcactgctgcctatgaccgccaacagctctggaaagccaacgtcggctttgttatccagctccaggcccgcctccgtggcttcctagttcggcagaagtttgctgagcattcccactttctgaggacctggctcccagcagtcatcaagatccaggctcattggcggggttataggcagcggaagatttacctggagtggttgcagtattttaaagcaaacctggatgccataatcaagatccaggcctgggcccggatgtgggcagctcggaggcaatacctgaggcgtctgcactacttccagaagaatgttaactccattgtgaagatccaggcatttttccgagccaggaaagcccaagatgactacaggatattagtgcatgcaccccaccctcctctcagtgtggtacgcagatttgcccatctcttgaatcaaagccagcaagacttcttggctgaggcagagctgctgaagctccaggaagaggtagttaggaagatccgatccaatcagcagctggagcaggacctcaacatcatggacatcaagattggcctgctggtgaagaaccggatcactctgcaggaagtggtctcccactgcaagaagctgaccaagaggaataaggaacagctgtcagatatgatggttctggacaagcagaagggtttaaagtcgctgagcaaagagaaacggcagaaactagaagcataccaacacctcttctacctgctccagactcagcccatctacctggccaagctgatctttcagatgccacagaacaaaaccaccaagttcatggaggcagtgattttcagcctgtacaactatgcctccagccgccgagaggcctatctcctgctccagctgttcaagacagcactccaggaggaaatcaagtcaaaggtggagcagccccaggacgtggtgacaggcaacccaacagtggtgaggctggtggtgagattctaccgtaatgggcggggacagagtgccctgcaggagattctgggcaaggttatccaggatgtgctagaagacaaagtgctcagcgtccacacagaccctgtccacctctataagaactggatcaaccagactgaggcccagacagggcagcgcagccatctcccatatgatgtcaccccggagcaggccttgagccaccccgaggtccagagacgactggacatcgccctacgcaacctcctcgccatgactgataagttccttttagccatcacctcatctgtggaccaaattccgtatgggatgcgatatgtggccaaagtcctgaaggcaactctggcagagaaattccctgacgccacagacagcgaggtctataaggtggtcgggaacctcctgtactaccgcttcctgaacccagctgtggtggctcctgacgccttcgacattgtggccatggcagctggtggagccctggctgccccccagcgccatgccctgggggctgtggctcagctcctacagcacgctgcggctggcaaggccttctctgggcagagccagcacctacgggtcctgaatgactatctggaggaaacacacctcaagttcaggaagttcatccatagagcctgccaggtgccagagccagaggagcgttttgcagtggacgagtactcagacatggtggctgtggccaaacccatggtgtacatcaccgtgggggagctggtcaacacgcacaggctgttgctggagcaccaggactgcattgcccctgatcaccaagaccccctgcatgagctcctggaggatcttggggagctgcccaccatccctgaccttattggtgagagcatcgctgcagatgggcacacggacctgagcaagctagaagtgtccctgacgctgaccaacaagtttgaaggactagaggcagatgctgatgactccaacacccgtagcctgcttctgagcaccaagcagctgttggccgatatcatacagttccatcctggggacaccctcaaggagatcctgtccctctcggcttccagagagcaagaagcagcccacaagcagctgatgagccgacgccaggcctgtacagcccagacaccggagccactgcgacgacaccgctcactgacagctcactccctcctgccactggcagagaagcagcggcgcgtcctgcggaacctacgccgacttgaagccctggggttggtcagcgccagaaatggctaccaggggctagtggacgagctggccaaggacatccgcaaccagcacagacacaggcacaggcggaaggcagagctggtgaagctgcaggccacattacagggcctgagcactaagaccaccttctatgaggagcagggtgactactacagccagtacatccgggcctgcctggaccacctggcccccgactccaagagttctgggaaggggaagaagcagccttctcttcattacactgctgctcagctcctggaaaagggtgtcttggtggaaattgaagatcttcccgcctctcacttcagaaacgtcatctttgacatcacgccgggagatgaggcaggaaagtttgaagtaaatgccaagttcctgggtgtggacatggagcgatttcagcttcactatcaggatctcctgcagctccagtatgagggtgtggctgtcatgaaactcttcaacaaggccaaagtcaatgtcaaccttctcatcttcctcctcaacaagaagtttttgcggaagtgacagaggcaaagggtgctacccaagcccctcttacctctctggatgctttctttaacactaactcaccactgtgcttccctgcagacacccagagctcaggactgggcaaggcccagggattctcaccccttccccagctgggaggagcttgcctgcctggccacagacagtgtatcttctaattggctaaagtgggccttgcccagagtccagctgtgtggcttttatcatgcatgacaaacccctggctttcctgccagatggtaggacatggaccttgacctgggaaagccattactcttgtgtctgctactgccctcccacagtcaccccaatattacaagcactgccccagcggcttgatttcccctctgccttccttctctctgcactcccacaaagccagggccaggctccccatccctacctcccactgcatcagcagtgggtgttcctgcccttcctgagtctaggcagctctgctgctgtgatctgcacaccctccaacctgggcagggactggggggatgcagtgtgtgttagtgcccatgtggcattgtggcactgttgccccccatggcggcatgggcaagatgaccttccattagcttcaagtcttgttctcttgtctgtggtctgtttaatatgtgggtcactagggtatttattctttctcccatccttacactctggatcattgtgcagacttaatcagggttttaacgctttcatttttttttttttttttttttttttgagctcaaagagagttctcattttccctattcaaactaatacccatgccgtgttttttaccttggatttaaagtcaccttaggttggggcaacagattctcactcatgtttaagatcttgttatttcagcttcataagatcaaagaggagtctttcccttttctcttttaccctcaggattctcatcccttacagctgactcttccaggcaatttccatagatctgcagtcctgcctctgccacagtctctctgttgtccccacatctacccaacttcctgtactgttgcccttctgatgttaataaaagcagctgttactcccaaaaaaaaaaaaaaaaaa

SEQ ID NO: 154 - protein, 1631 aa, Homo sapiens
MERRAAGPGWAAYERLTAEEMDEQRRQNVAYQYLCRLEEAKRWMEACLKEELPSPVELEESLRNGVLLAKLGHCFAPSVVPLKKIYDVEQLRYQATGLHFRHTDNINFWLSAIAHIGLPSTFFPETTDIYDKKNMPRVVYCIHALSLFLFRLGLAPQIHDLYGKVKFTAEELSNMASELAKYGLQLPAFSKIGGILANELSVDEAAVHAAVLAINEAVERGVVEDTLAALQNPSALLENLREPLAAVYQEMLAQAKMEKAANARNHDDRESQDIYDHYLTQAEIQGNINHVNVHGALEVVDDALERQSPEALLKALQDPALALRGVRRDFADWYLEQLNSDREQKAQELGLVELLEKEEVQAGVAAANTKGDQEQAMLHAVQRINKAIRRGVAADTVKELMCPEAQLPPVYPVASSMYQLELAVLQQQQGELGQEELFVAVEMLSAVVLINRALEARDASGFWSSLVNPATGLAEVEGENAQRYFDALLKLRQERGMGEDFLSWNDLQATVSQVNAQTQEETDRVLAVSLINEALDKGSPEKTLSALLLPAAGLDDVSLPVAPRYHLLLVAAKRQKAQVTGDPGAVLWLEEIRQGVVRANQDTNTAQRMALGVAAINQAIKEGKAAQTERVLRNPAVALRGVVPDCANGYQRALESAMAKKQRPADTAFWVQHDMKDGTAYYFHLQTFQGIWEQPPGCPLNTSHLTREEIQSAVTKVTAAYDRQQLWKANVGFVIQLQARLRGFLVRQKFAEHSHFLRTWLPAVIKIQAHWRGYRQRKIYLEWLQYFKANLDAIIKIQAWARMWAARRQYLRRLHYFQKNVNSIVKIQAFFRARKAQDDYRILVHAPHPPLSVVRRFAHLLNQSQQDFLAEAELLKLQEEVVRKIRSNQQLEQDLNIMDIKIGLLVKNRITLQEVVSHCKKLTKRNKEQLSDMMVLDKQKGLKSLSKEKRQKLEAYQHLFYLLQTQPIYLAKLIFQMPQNKTTKFMEAVIFSLYNYASSRREAYLLLQLFKTALQEEIKSKVEQPQDVVTGNPTVVRLVVRFYRNGRGQSALQEILGKVIQDVLEDKVLSVHTDPVHLYKNWINQTEAQTGQRSHLPYDVTPEQALSHPEVQRRLDIALRNLLAMTDKFLLAITSSVDQIPYGMRYVAKVLKATLAEKFPDATDSEVYKVVGNLLYYRFLNPAVVAPDAFDIVAMAAGGALAAPQRHALGAVAQLLQHAAAGKAFSGQSQHLRVLNDYLEETHLKFRKFIHRACQVPEPEERFAVDEYSDMVAVAKPMVYITVGELVNTHRLLLEHQDCIAPDHQDPLHELLEDLGELPTIPDLIGESIAADGHTDLSKLEVSLTLTNKFEGLEADADDSNTRSLLLSTKQLLADIIQFHPGDTLKEILSLSASREQEAAHKQLMSRRQACTAQTPEPLRRHRSLTAHSLLPLAEKQRRVLRNLRRLEALGLVSARNGYQGLVDELAKDIRNQHRHRHRRKAELVKLQATLQGLSTKTTFYEEQGDYYSQYIRACLDHLAPDSKSSGKGKKQPSLHYTAAQLLEKGVLVEIEDLPASHFRNVIFDITPGDEAGKFEVNAKFLGVDMERFQLHYQDLLQLQYEGVAVMKLFNKAKVNVNLLIFLLNKKFLRK